SEQUENCE LISTING (19 sequences)

SEQ ID NO: 1 - DNA, 20 bp, Artificial
  Primer
aaatgaattgctcaacattc

SEQ ID NO: 2 - DNA, 22 bp, Artificial
  Primer
gtagataattaatgatgacgcg

SEQ ID NO: 3 - DNA, 20 bp, Artificial
  Primer
gggagcatacatagtatggg

SEQ ID NO: 4 - DNA, 18 bp, Artificial
  Primer
atgaagtgctcaacattc

SEQ ID NO: 5 - DNA, 22 bp, Artificial
  Primer
taagatcattaatgacgatgcc

SEQ ID NO: 6 - DNA, 19 bp, Artificial
  Primer
atacacagtacatccggac

SEQ ID NO: 7 - DNA, 20 bp, Artificial
  Primer
ctaaagtgtccacctacgcc

SEQ ID NO: 8 - DNA, 1638 bp, Cannabis sativa
atgaattgctcaacattctccttttggtttgtttgcaaaataatgtttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctctaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaa

SEQ ID NO: 9 - protein, 545 aa, Cannabis sativa
MNCSTFSFWFVCKIMFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 10 - DNA, 1638 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaa

SEQ ID NO: 11 - protein, 545 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 12 - DNA, 45 bp, Artificial
  Primer
agcaagttcttcactgttgatacataaatgaattgctcaacattc

SEQ ID NO: 13 - DNA, 47 bp, Artificial
  Primer
gagttgttgattcagaattgtcgacgtagataattaatgatgacgcg

SEQ ID NO: 14 - DNA, 21 bp, Artificial
  Primer
ctatccttcgcaagacccttc

SEQ ID NO: 15 - DNA, 23 bp, Artificial
  Primer
caaacttaagcacacaagctagc

SEQ ID NO: 16 - DNA, 28 bp, Artificial
  Primer
ctccttttggtttgtttgcaaaatactg

SEQ ID NO: 17 - DNA, 24 bp, Artificial
  Primer
cgaatctgcaaaccaactttcgta

SEQ ID NO: 18 - DNA, 28 bp, Artificial
  Primer
ctccttttggtttgtttgcaaaataata

SEQ ID NO: 19 - DNA, 24 bp, Artificial
  Primer
cgaatctgcaaaccaactttcttg

(19) United States
(12) Patent Application Publication
(10) Pub. No.: US 2023/0413750 A1
(43) Pub. Date: Dec. 28, 2023

(54) CANNABIS PLANT WITH INCREASED CANNABICHROMENIC ACID

(71) Applicant: CANOPY GROWTH CORPORATION, Smiths Falls (CA)

(72) Inventor: Daniel Kevin FOWLER, Arvada, CO (US)

(21) Appl. No.: 18/248,774

(22) PCT Filed: Oct. 13, 2021
(86) PCT No.: PCT/US2021/054744

§ 371 Date: Apr. 12, 2023

Related U.S. Application Data
(60) Provisional application No. 63/091,057, filed on Oct. 13, 2020.

(51) Int. Cl. A01H 1/00; A01H 6/28; A01H 5/02; C12N 9/02

(52) U.S. Cl. CPC A01H 1/101; A01H 6/28; A01H 5/02; C12N 9/0004

(57) ABSTRACT

The present technology generally relates to Cannabis plants having increased cannabichromenic acid (CBCA) and/or cannabichromene (CBC) content as well as to nucleic acids related to same and methods of producing same.

### CROSS-REFERENCE TO RELATED APPLICATION

This application claims the benefit of and priority to U.S. provisional patent application No. 63/091,057, filed on Oct. 13, 2020; the content of which is herein incorporated in entirety by reference.

### FIELD OF TECHNOLOGY

The present technology generally relates to Cannabis plants having increased cannabichromenic acid (CBCA) and/or cannabichromene (CBC) content as well as to nucleic acids related to same and methods of producing same.

### BACKGROUND INFORMATION

Cannabis is a genus of flowering plants that produce a unique a class of terpenophenolic compounds known as cannabinoids. Cannabinoids interact with receptors of human and animal endocannabinoid systems and can lead to a plethora of potential medical and therapeutic effects (Di Marzo & Piscitelli, 2015). In the Cannabis plant's biosynthetic pathway, cannabigerolic acid (CBGA) is the first cannabinoid produced by enzymatic condensation of olivetolic acid and geranyl pyrophosphate (Gagne et al., 2012). CBGA is then converted to the three terminal cannabinoids Δ9-tetrahydrocannabinolic acid (THCA), cannabidiolic acid (CBDA) and cannabichromenic acid (CBCA) by the enzymes THCA synthase (THCAS), CBDA synthase (CBDAS) and CBCA synthase (CBCAS), respectively (Laverty et al., 2019) (FIG. 1). Non-enzymatic decarboxylation of the cannabinoids, primarily through heating, produces the neutral forms cannabigerol (CBG), Δ9-tetrahydrocannabinol (THC), cannabidiol (CBD) and cannabichromene (CBC).

THC, the major intoxicating cannabinoid responsible for cannabis' “high” when inhaled or ingested, and CBD, a non-intoxicating cannabinoid are the two most prevalent cannabinoids in individual cannabis cultivars, known colloquially as “strains,” and have been extensively studied for human and animal health purposes (Lewis et al., 2018). However over 70 other “rare” cannabinoids have been found in cannabis plant samples, many of which have promising pharmacological activity (ElSohly & Slade, 2005). CBC (produced as CBCA in planta), typically found in trace amounts in most cannabis strains, is one such cannabinoid.

Many peer-reviewed studies have suggested multiple medical uses for CBC and CBCA. Rodent studies have shown CBC is an analgesic (DeLong et al., 2010) (Davis & Hatoum, 1983) (Maione et al., 2011), a potent anti-inflammatory drug both in vitro and in vivo (Romano et al., 2013) (Izzo et al., 2012) (C. E. Turner & Elsohly, 1981) (Wirth et al., 1980), and an antidepressant (El-Alfy et al., 2010). In vitro studies showed that CBC enhanced the viability of mouse neural stem progenitor cells (Shinjyo & Di Marzo, 2013) and a meta-analysis of published studies suggested CBC was promising as a neuroprotectant in seizure, hypomobility, Huntington's and Parkinson's disease (Stone et al., 2020).

CBC has also been demonstrated to be a potent antibacterial (C. E. Turner & Elsohly, 1981), including against methicillin-resistant Staphylococcus aureus (MRSA) (Appendino et al., 2008). Recently, CBCA was found to be a more potent antibiotic against MRSA than first-line treatment antibiotic vancomycin, while maintaining mammalian cell viability (Galletta et al., 2020).

Because it is typically a rare cannabinoid, there is a need to develop Cannabis strains which produce higher cannabichromenic acid (CBCA) and/or cannabichromene (CBC) levels.

### SUMMARY OF DISCLOSURE

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 1% of total dry flower weight.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 2% of total dry flower weight.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 3% of total dry flower weight.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 4% of total dry flower weight.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 5% of total dry flower weight.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content of between about 1% and about 10% of total dry flower weight.

According to various aspects, the present technology relates to an isolated nucleic acid molecule comprising an expression-altering variant cannabichromenic acid (CBCA) synthase (CBCAS) allele, wherein the expression-altering variant CBCAS allele comprises an expression-altering variation that causes expression or an increase in expression of CBCAS.

According to various aspects, the present technology relates to an isolated nucleic acid molecule comprising a nucleotide sequence having at least, greater than or about 75% sequence identity to SEQ ID NO: 8, wherein the nucleic acid sequence comprises an expression-altering variation.

According to various aspects, the present technology relates to an isolated nucleic acid molecule comprising a nucleotide sequence having at least, greater than or about 85% sequence identity to SEQ ID NO: 8, wherein the nucleic acid sequence comprises an expression-altering variation.

According to various aspects, the present technology relates to an isolated nucleic acid molecule having a nucleic acid sequence as set forth in any one of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 12; SEQ ID NO: 13, SEQ ID NO: 16, SEQ ID NO: 17, SEQ ID NO: 18 and SEQ ID NO: 19 or a complementary nucleic acid sequence thereof.

According to various aspects, the present technology relates to an isolated DNA marker for identifying an expression-altering variant cannabichromenic acid synthase (CBCAS) allele in a plant, the isolated DNA marker comprising SEQ ID NO: 8 or a fragment thereof comprising an expression-altering variation.

According to various aspects, the present technology relates to an isolated polypeptide encoded by the isolated nucleic acid molecule as defined herein.

According to various aspects, the present technology relates to an isolated polypeptide comprising an amino acid sequence as set forth in SEQ ID NO: 9 or a fragment thereof or analog thereof, wherein the isolated polypeptide comprises an expression-altering variation.

According to various aspects, the present technology relates to a cDNA molecule that codes for the isolated polypeptide as defined herein.

According to various aspects, the present technology relates to an antibody that specifically binds the isolated polypeptide as defined herein. According to various aspects, the present technology relates to an organism, tissue or cell comprising the isolated nucleic molecule and/or the isolated polypeptide as defined herein.

According to various aspects, the present technology relates to a method for increasing levels of cannabichromenic acid (CBCA) and/or cannabichromene (CBC) in a tissue or a cell, the method comprising introducing an expression-altering variation in the nucleic acid sequence encoding for cannabichromenic acid synthase (CBCAS) in the tissue or the cell.

According to various aspects, the present technology relates to a method of increasing levels of cannabichromenic acid (CBCA) and/or cannabichromene (CBC) in a tissue or a cell, the method comprising: a) introducing into a tissue or a cell: i) a nucleic acid sequence comprising SEQ ID NO: 8 or a fragment thereof, the fragment thereof retaining an expression-altering variation; or ii) a nucleic acid sequence having at least or about 75% sequence identity to SEQ ID NO: 8 while retaining the expression-altering variation; to produce a recombinant tissue or recombinant cell; b) culturing the recombinant tissue or the recombinant cell under conditions that permit expression of the nucleic acid.

According to various aspects, the present technology relates to a Cannabis plant comprising a variant cannabichromenic acid synthase (CBCAS) allele, wherein the expression-altering variant CBCAS allele encodes for CBCAS.

According to various aspects, the present technology relates to a Cannabis plant comprising an expression-altering variant cannabichromenic acid synthase (CBCAS) allele, wherein the plant expresses a CBCAS transcript.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 1% of total dry flower weight and comprises the isolated nucleic acid as defined herein.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 2% of total dry flower weight and comprises the isolated nucleic acid as defined herein.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 3% of total dry flower weight and comprises the isolated nucleic acid as defined herein.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 4% of total dry flower weight and comprises the isolated nucleic acid as defined herein.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content greater than about 5% of total dry flower weight and comprises the isolated nucleic acid as defined herein.

According to various aspects, the present technology relates to a Cannabis plant, plant part, tissue or cell thereof, wherein the Cannabis plant, plant part, tissue or cell thereof has a cannabichromenic acid (CBCA) and/or cannabichromene (CBC) content of between about 1% and about 25% of total dry flower weight and comprises the isolated nucleic acid as defined herein.

Other aspects and features of the present disclosure will become apparent to those ordinarily skilled in the art upon review of the following description of specific embodiments.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a diagram showing the biosynthesis pathway for tetrahydrocannabinolic acid (THCA), cannabidiolic acid (CBDA) and cannabichromenic acid (CBCA) from the precursor cannabigerolic acid (CBGA);

FIG. 2 shows a high-performance liquid chromatography (HPLC) chromatogram, calculated cannabinoid contents, and total THC, CBD, and CBC contents by dry weight for de-seeded flower material from CBC-enriched strain CGC1 at 12 weeks post flower initiation;

FIG. 3 shows graphs for average total CBD (top panel) and total CBC (middle panel) content by dry weight analyzed by HPLC and total CBD:CBC ratio (bottom panel) from CBC-enriched hemp strain CGC1 and non-CBC enriched hemp strains CGC2 and CGC3, sampled weekly across 3 to 4 plants for each strain, from 3 to 6 weeks post flower initiation. Error bars indicate standard error. Asterisks indicate significant differences between measurements for CGC1 and both CGC2 and CGC3 by two-tailed Student's t-test (*P≤0.05, **P≤0.01, ***P≤0.001);

FIG. 4 is a schematic representation showing polymerase chain reaction (PCR) oligonucleotide primers used to amplify CBCAS and CBDAS protein-coding regions. CBCAS and CBDAS are structurally similar intronless genes with predicted messenger RNA (mRNA) transcripts containing a 5′ untranslated region (UTR), open reading frame (ORF) and 3′UTR identical to the genomic sequence. Primers specific to CBCAS or CBDAS sequences were used with a forward primer anchored near the beginning start codon of the open reading frame (ORF forward) and with reverse primers either anchored near the stop codon of the ORF (ORF reverse) or in the 3′UTR (3′UTR reverse);

FIG. 5 shows photographs of gels of PCR results from complimentary DNA (cDNA) templates derived from total RNA isolated from trichomes of flowers at 6 weeks post flower initiation (left panel), or genomic DNA (gDNA) (right panel) isolated from vegetative leaves from CBC-enriched hemp strain CGC1 and non-CBC enriched hemp strains CGC2 and CGC3. Note that only CBCAS amplified from trichome-derived cDNA from CBC-enriched strain CGC1 although CBCAS was detected in the genome of all three strains. Because all three strains are CBD/CBDA-dominant, CBDAS amplification served as a positive control for cDNA synthesis from trichome messenger RNA (mRNA) and was detected in all three strains;

FIG. 6 shows a nucleic acid sequence alignment between the expressed CBCAS messenger RNA sequence (mRNA) found exclusively in CBC-enriched hemp strain CGC1 (CBCASexpressed), the closest similarity CBCAS sequence to CBCASexpressed found across all Cannabis sequences deposited on the National Center for Biotechnology Information (NCBI) (CBCASclosestNCBI), and the CBCAS sequence disclosed in U.S. Pat. No. 10,364,4106 (CBCASUS103644106). Note the presence of single nucleotide polymorphisms (SNPs) A45G and C300T unique to CBCASexpressed;

FIG. 7 shows a translated amino sequence alignment between the expressed CBCAS messenger RNA sequence (mRNA) found exclusively in CBC-enriched hemp strain CGC1 (CBCASexpressed), the closest similarity CBCAS sequence to CBCASexpressed found across all Cannabis sequences deposited on the National Center for Biotechnology Information (NCBI) (CBCASclosestNCBI), and the CBCAS sequence disclosed in U.S. Pat. No. 10,364,4106 (CBCASUS103644106). Note the presence of amino acid change I15M unique to CBCASexpressed;

FIG. 8 shows Sanger sequencing chromatograms expanded at nucleotide positions 41-51 of CBCAS that was PCR amplified from genomic DNA templates for CBC-enriched hemp strain CGC1 and non-CBC enriched hemp strains CGC2 and CG3. The arrow demarks a low-abundance A45G SNP observed only in CGC1;

FIG. 9 shows a phylogenetic tree of all CBCAS alleles found in CGC1, CGC2 and CGC3 by clonal sequencing analysis from genomic DNA as well as CBCASexpressed and CBCASclosestNCBI sequences. The phylogeny was rooted on the sequence for CBCASclosestNCBI and the arrow demarks the CBCASexpressed sequence was only found in the genome of CGC1 (clone CGC1-11). Sequences aligned on the same vertical line indicate identical nucleotide sequences;

FIG. 10 is a photograph of a gel showing PCR results from genomic DNA (gDNA) of CBC-enriched hemp strain CGC1 and non-CBC enriched hemp strains CGC2 and CGC3 using primers specific for CBCASexpressed or CBCASclosestNCBI sequences. Note that CBCASexpressed was only detected in gDNA from CGC1;

FIG. 11 shows whole flower photographs and two different magnification level photographs of flowers for CBC-enriched strain CGC1 and non-CBC enriched strain CGC2 at 7 weeks post flower induction showing the near-complete absence of sessile (non-stalked) glandular trichomes in both strains;

FIG. 12 is a photograph of a gel showing the PCR results from genomic DNA (gDNA) of CBC-enriched parental strain CGC1 and a parental hemp strain that was CBG-dominant and 15 of their F1 progeny using primers specific for CBCASexpressed or CBCASclosestNCBI sequences. CBCASexpressed was detected in parent CGC1 but not the CBG-dominant parent;

FIG. 13 is a photograph of a gel showing the PCR results from genomic DNA (gDNA) of CBC-enriched parental strain CGC1 and 30 of its selfed (S1) progeny using primers specific for CBCASexpressed or CBCASclosestNCBI sequences; and

FIG. 14 shows graphs for average total CBD (left panel) and total CBC (middle panel) content by dry weight analyzed by HPLC and single plant data points for CBD:CBC ratio (right panel) from two replicates of CBC-enriched parental hemp strain CGC1 and 22 of its selfed (S1) progeny at 7 weeks post flower initiation. Error bars indicate standard error. Asterisks indicate significant differences between groups by two-tailed Student's t-test (*P≤0.05, **P≤0.01, ***P≤0.001, n.s.; not significant).

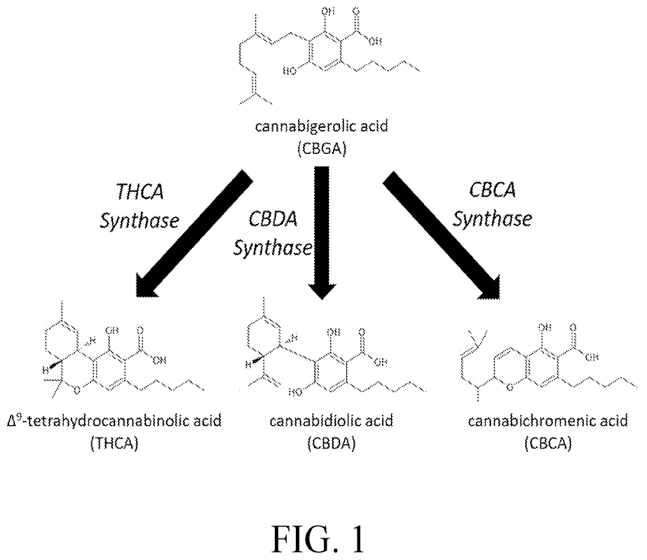

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure in which variations and additions do not depart from the present technology. Hence, the following description is intended to illustrate some particular embodiments of the technology, and not to exhaustively specify all permutations, combinations and variations thereof.

As used herein, the singular form “a,” “an”, and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein explicitly or not. Every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “Cannabis” refers to the genus of flowering plants in the family Cannabaceae regardless of species, subspecies, or subspecies variety classification. At present, there is no general consensus whether plants of genus Cannabis are comprised of a single or multiple species (McPartland & Guy, 2017). For example some describe Cannabis plants as a single species, C. sativa L., with multiple subspecies (Small & Cronquist, 1976) (McPartland & Small, 2020) while others classify Cannabis plants into multiple species, most commonly as C. sativa L. and C. indica Lam. and sometimes additionally as C. ruderalis Janisch. (Schultes et al., 1974), depending on multiple criteria including morphology, geographic origin, chemical content, and genetic measurements. Regardless, all plants of genus Cannabis can interbreed and produce fertile offspring (Small, 1972)

The term “strain” as used herein refers to different varieties of the plant genus Cannabis. For example, the term “strain” can refer to different pure or hybrid varieties of Cannabis plants. In some instances, the Cannabis strain of the present technology can by a hybrid of two strains. Different Cannabis strains often exhibit distinct chemical compositions with characteristic levels of cannabinoids and terpenes, as well as other components. Differing cannabinoid and terpene profiles associated with different Cannabis strains can be useful for the treatment of different diseases, or for treating different subjects with the same disease.

As used herein, the term “cannabinoid” refers to a chemical compound belonging to a class of secondary compounds commonly found in plants of genus Cannabis, but also encompasses synthetic and semi-synthetic cannabinoids and any enantiomers thereof. In an embodiment, the cannabinoid is a compound found in a plant, e.g., a plant of genus Cannabis, and is sometimes referred to as a phytocannabinoid. In one embodiment, the cannabinoid is a compound found in a mammal, sometimes called an endocannabinoid. In one embodiment, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids.

In another embodiment, a cannabinoid is one of a class of diverse chemical compounds that may act on cannabinoid receptors such as CB₁ and CB₂ in cells that alter neurotransmitter release in the brain.

In many cases, a cannabinoid can be identified because its chemical name will include the text string “*cannabi*”. However, there are a number of cannabinoids that do not use this nomenclature, such as for example those described herein.

As used herein, the expression “% by weight” is calculated based on dry weight of the total material.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids. As well, any and all isomeric, enantiomeric, or optically active derivatives are also encompassed. In particular, where appropriate, reference to a particular cannabinoid incudes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

In some embodiments of the present disclosure, the cannabinoid is a cannabinoid dimer. The cannabinoid may be a dimer of the same cannabinoid (e.g. THC-THC) or different cannabinoids. In an embodiment of the present disclosure, the cannabinoid may be a dimer of THC, including for example Cannabisol.

In an embodiment, a cannabinoid may occur in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms.

As used herein, the expressions “nucleic acid,” “nucleic acid molecule,” “oligonucleotide,” and “polynucleotide” are each used herein to refer to a polymer of at least three nucleotides. In some embodiments, a nucleic acid comprises deoxyribonucleic acid (DNA). In some embodiments comprises ribonucleic acid (RNA). In some embodiments, a nucleic acid is single stranded. In some embodiments, a nucleic acid is double stranded. In some embodiments, a nucleic acid comprises both single and double stranded portions. Unless otherwise stated, the terms encompass nucleic acid-like structures with synthetic backbones, as well as amplification products. In some embodiments, nucleic acids of the present disclosure are linear nucleic acids.

As used herein, the term “gene” refers to a part of the genome that code for a product (e.g., an RNA product and/or a polypeptide product). A “gene sequence” is a sequence that includes at least a portion of a gene (e.g., all or part of a gene) and/or regulatory elements associated with a gene. In some embodiments, a gene includes coding sequence; in some embodiments, a gene includes non-coding sequence. In some particular embodiments, a gene may include both coding (e.g., exonic) and non-coding (e.g., intronic) sequences. In some embodiments, a gene may include one or more regulatory elements (e.g., a promoter) that, for example, may control or impact one or more aspects of gene expression (e.g., cell-type-specific expression, inducible expression, etc.).

As used herein, the expression “coding sequence” refers to a sequence of a nucleic acid or its complement, or a part thereof, that: i) can be transcribed to an mRNA sequence that can be translated to produce a polypeptide or a fragment thereof, or ii) an mRNA sequence that can be translated to produce a polypeptide or a fragment thereof. Coding sequences include exons in genomic DNA or immature primary RNA transcripts, which are joined together by the cell's biochemical machinery to provide a mature mRNA.

As used herein, the term “mutation” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence, or the increase or reduction/elimination of an existing character, property, function, phenotype or trait not found in the protein encoded by the parental sequence.

The expression “degree or percentage of sequence homology” refers herein to the degree or percentage of sequence identity between two sequences after optimal alignment. Percentage of sequence identity (or degree of identity) is determined by comparing two aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

As used herein, the term “isolated” refers to nucleic acids or polypeptides that have been separated from their native environment, including but not limited to virus, proteins, glycoproteins, peptide derivatives or fragments or polynucleotides. For example, the expression “isolated nucleic acid molecule” as used herein refers to a nucleic acid substantially free of cellular material or culture medium when produced by recombinant DNA techniques, or chemical precursors, or other chemicals when chemically synthesized. An isolated nucleic acid is also substantially free of sequences, which naturally flank the nucleic acid (i.e. sequences located at the 5′ and 3′ ends of the nucleic acid) from which the nucleic acid is derived.

Two nucleotide sequences or amino-acids are said to be “identical” if the sequence of nucleotide residues or amino-acids in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482 (1981), by the homology alignment algorithm of Needleman and Wunsch, J. Mol. Biol. 48: 443 (1970), by the search for similarity method of Pearson and Lipman, Proc. Natl. Acad. Sci. (U.S.A.) 85: 2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection. Other alignment programs may also be used such as: “Multiple sequence alignment with hierarchical clustering”, F. CORPET, 1988, Nucl. Acids Res., 16 (22), 10881-10890.

As used herein, the expression “conservative substitutions” refers to a substitution made in an amino acid sequence of a polypeptide without disrupting the structure or function of the polypeptide. Conservative amino acid substitutions may be accomplished by substituting amino acids with similar hydrophobicity, polarity, and R-chain length for one another. Additionally, by comparing aligned sequences of homologous proteins from different species, conservative amino acid substitutions may be identified by locating amino acid residues that have been mutated between species without altering the basic functions of the encoded proteins. Amino acid substitutions that are conservative are typically as follows: i) hydrophilic: Alanine (Ala) (A), Proline (Pro) (P), Glycine (Gly) (G), Glutamic acid (Glu) (E), Aspartic acid (Asp) (D), Glutamine (Gln) (Q), Asparagine (Asn) (N), Serine (Ser) (S), Threonine (Thr) (T); ii) Sulphydryl: Cysteine (Cys) (C); iii) Aliphatic: Valine (Val) (V), Isoleucine (Ile) (I), Leucine (Leu) (L), Methionine (Met) (M); iv) Basic: Lysine (Lys) (K), Arginine (Arg) (R), Histidine (His) (H); and v) Aromatic: Phenylalanine (Phe) (F), Tyrosine (Tyr) (Y), Tryptophan (Trp) (W).

An “expression system” as used herein refers to reagents and components (e.g. in a kit) and/or solutions comprising said reagents and components for recombinant protein expression, wherein the expression system is cell free and includes optionally translation competent extracts of whole cells and/or other translation machinery reagents or components optionally in a solution, said reagents and components optionally including RNA polymerase, one or more regulatory protein factors, one or more transcription factors, ribosomes, and tRNA, optionally supplemented with cofactors and nucleotides, and the specific gene template of interest. Chemical based expression systems are also included, optionally using unnaturally occurring amino acids. In some instances, the expression systems of the present technology are in vitro expression system.

The expressions “transformed with”, “transfected with”, “transformation” and “transfection” are intended to encompass introduction of nucleic acid (e.g. a construct) into a cell by one of many possible techniques known in the art.

The term “primer” as used herein, typically refers to oligonucleotides that hybridize in a sequence specific manner to a complementary nucleic acid molecule (e.g., a nucleic acid molecule comprising a target sequence). In some embodiments, a primer will comprise a region of nucleotide sequence that hybridizes to at least 8, e.g., at least 10, at least 15, at least 20, at least 25, or 20 to 60 nucleotides of a target nucleic acid (i.e., will hybridize to a sequence of the target nucleic acid). In general, a primer sequence is identified as being either “complementary” (i.e., complementary to the coding or sense strand (+)), or “reverse complementary” (i.e., complementary to the anti-sense strand (−)). In some embodiments, the term “primer” may refer to an oligonucleotide that acts as a point of initiation of a template-directed synthesis using methods such as PCR (polymerase chain reaction) under appropriate conditions (e.g., in the presence of four different nucleotide triphosphates and a polymerization agent, such as DNA polymerase in an appropriate buffer solution containing any necessary reagents and at suitable temperature(s)). Such a template directed synthesis is also called “primer extension.” For example, a primer pair may be designed to amplify a region of DNA using PCR. Such a pair will include a “forward primer” and a “reverse primer” that hybridize to complementary strands of a DNA molecule and that delimit a region to be synthesized and/or amplified.

As used herein, the expression “wild-type” refers to a typical or common form existing in nature; in some embodiments it is the most common form.

The term “antibody” as used herein is intended to include monoclonal antibodies, polyclonal antibodies, and chimeric antibodies. The antibody may be from recombinant sources and/or produced in transgenic animals. Antibody binding fragment: The term “antibody binding fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. Antibodies may be monospecific, bispecific, trispecific or of greater multispecificity. Multispecific antibodies may immunospecifically bind to different epitopes of a cannabichromenic acid synthase and/or or a solid support material. Antibodies may be prepared using methods known to those skilled in the art. Isolated native or recombinant polypeptides may be utilized to prepare antibodies. See, for example, Kohler et al. (1975) Nature 256:495-497; Kozbor et al. (1985) J. Immunol. Methods, 81:31-42; Cote et al. (1983) Proc Natl Acad Sci., 80:2026-2030; and Cole et al. (1984) Mol Cell Biol., 62:109-120, for the preparation of monoclonal antibodies; Huse et al. (1989) Science, 246:1275-1281, for the preparation of monoclonal Fab fragments; and, Pound (1998) Immunochemical Protocols, Humana Press, Totowa, N.J., for the preparation of phagemid or B-lymphocyte immunoglobulin libraries to identify antibodies.

As used herein, the expression “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots.” Plant part may also include certain extracts such as kief or hash which includes Cannabis trichomes or glands.

As used herein, the term “chemotype” refers to the cannabinoid chemical phenotype in individual Cannabis strains. In general, chemotype is primarily determined by, but not limited to, chemical ratios or predominance of CBD, THC, CBC, and CBG and/or their acid counterparts CBDA, THCA, CBCA, and CBGA present in mature or semi-mature Cannabis flower.

As used herein, the term “total cannabinoid” (e.g. “total CBC”) refers to a neutral cannabinoid content+(corresponding acidic cannabinoid content*0.877) (e.g. CBC content+(CBCA content*0.877) in a cannabis plant part.

As used herein, the term “trace” when referring to cannabinoid content generally refers to a total cannabinoid content of less than about 0.5% by dry weight in mature or semi-mature Cannabis flower or a cannabis plant part.

As used herein, the term “CBC-enriched” refers to greater than about 1% by dry weight total CBC and/or a total CBD:CBC ratio of equal to or less than about 15:1 in mature or semi-mature Cannabis flower or cannabis plant part.

As used herein, the term “CBC-dominant” refers to a total CBC content greater than about 50% of the total content of all cannabinoids measured in mature or semi-mature Cannabis flower or cannabis plant part.

During early cannabis research, chemotypes were largely based on ratios of the two most abundant cannabinoids: THCA and CBDA (Small & Beckstead, 1973). Plants producing primarily THCA are Type I, primarily producing CBDA are Type III, or producing significant amounts of both THCA and CBDA are Type II. Levels of minor cannabinoids were usually not accounted for in chemotype analyses.

Following this several reports were made during the 1970s and 1980s of cannabis from various geographical origins, usually showing trace levels of CBC in both THC and CBD-dominant varieties (Carlton E. Turner & Hadley, 1973a) (Baker et al., 1983) but some showing enriched or even dominant levels of CBC (Shoyama et al., 1975) (ROWAN & FAIRBAIRN, 1977). However, scrutiny must be applied to these claims of high-CBC varieties. For example, samples other than flowering material (e.g. seedlings or vegetative leaves) were typically used for analysis and it has since been demonstrated that in some cannabis varieties CBC can predominate THC and CBD levels at these vegetative stages but is quickly overtaken during the flowering stage (Vogelmann et al., 1988) (S. Morimoto et al., 1997) (E. P. M. De Meijer et al., 2009). Also it was difficult to separate peaks for CBD and CBC with the packed columns used for gas chromatography at the time suggesting the possibility of mistaking CBD abundance as CBC (Carlton E. Turner & Hadley, 1973b) (C. E. Turner et al., 1975). Therefore, it is difficult to determine if there was truly a cannabis variety isolated during this time period with a CBC-enriched or dominant flower chemotype.

A thorough analysis of cannabis flower chemotypes from a diverse germplasm collection was conducted years later which found CBC in trace amounts (but not enriched or dominant amounts) in most, but not all, plants across chemotypes I, II, and III (Hillig & Mahlberg, 2004). Another large and diverse survey of flower chemotypes found CBC in trace amounts for most varieties, but additionally found two strains with CBC-enriched or dominant chemotypes at maturity: one from an Afghan landrace with 58% cannabinoid fraction as CBC and a second from a Korean fiber hemp landrace with cannabinoid CBC fractions ranging from 7 to 58% (E. P. M. De Meijer et al., 2009) (US Patent application 20110098348 and 20160360721). However, these two high CBC strains were linked to a “prolonged juvenile characteristic” (PJC) as described in more detail below.

Besides these two strains identified by de Meijer et al. and their breeding derivatives (E. P. M. De Meijer et al., 2009) (US Patent application 20110098348 and 20160360721), it was reported that CBC levels have never been found at more than about 1-2% by dry weight in contemporary medical or recreational strains of cannabis (Hanus̆ et al., 2016) and CBC rarely exceeds 0.2-0.3% (Pollastro et al., 2018). Indeed, these claims were recently supported by the most comprehensive report to date of CBC concentrations from a subset of 17,600 cannabis flower samples tested in California, Colorado, and Washington states. In this study only a few samples tested over 1% CBC by dry weight, all were under 2%, and the vast majority contained 0-0.3% (Vergara et al., 2020). Collectively these results firmly demonstrate that modern cannabis with enriched or dominant concentrations of CBC are exceedingly rare.

The genetic basis of CBC production in cannabis remains unclear. Much more is known about the genetics for THC and CBD production. Based on thorough genetic inheritance studies, de Meijer et al. postulated that most cannabis strains contain a co-dominant cannabinoid synthase locus (B) that contains either THCAS (Bt) or CBDAS (Bd) (Etienne P. M. De Meijer et al., 2003). If functional versions of these enzymes are present they will convert CBGA into THCA or CBDA, respectively. Since Cannabis is diploid, plants homozygous for THCAS (Bt/Bt, Type I) will be dominant for THCA, plants homozygous for CBDAS (Bd/Bd, Type III) will be dominant for CBDA, and heterozygous plants (Bt/Bd, Type II) will produce significant amounts of both. This inheritance model has recently been confirmed by next-generation sequencing methods coupled with cross-breeding and chemotyping (Grassa et al., 2018) (Laverty et al., 2019).

Cannabinoids primarily accumulate in resin glands on the plant surface known as glandular trichomes. It was recently shown that sessile glandular trichomes (without a stalk) develop into stalked glandular trichomes during flowering in cannabis and this transition is highly correlated to cannabinoid accumulation (Livingston et al., 2020) (Mahlberg & Eun, 2004). For CBC production, de Meijer et al. postulated that CBCAS may be expressed in sessile trichomes during the vegetative state but shut off during the formation of stalked trichomes in favor of the expression of THCAS and/or CBDAS (E. P. M. De Meijer et al., 2009) (US Patent application 20110098348 and 20160360721). In agreement with this model, the CBC-dominant strains they developed had a complete absence of stalked glandular trichomes during flowering called a “prolonged juvenile characteristic” or PJC (E. P. M. De Meijer et al., 2009) (US Patent application 20110098348 and 20160360721). The lack of conversion from sessile to stalked trichomes during flower maturation also prevented significant cannabinoid accumulation such that total cannabinoid content in mature CBC-dominant flowers was no higher than about 3% by dry weight, because sessile glandular trichomes contain approximately 20-fold less total cannabinoids than mature stalked glandular trichomes (Mahlberg & Eun, 2004). The hypothesis that CBCAS is expressed in sessile trichomes but is turned off in stalked trichomes was not tested by de Meijer et. al because the gene had not been identified and the hypothesis remains uninvestigated to this date.

A putative CBCAS enzyme capable of converting CBGA to CBCA was isolated from cannabis plant extracts but no DNA sequence encoding this enzyme was isolated at the time (Satoshi Morimoto et al., 1998). Later, a separate group cloned and characterized a genomic DNA sequence for CBCAS in a high-THC cannabis strain (U.S. Pat. No. 10,364,416, incorporated herein by reference) (Laverty et al., 2019). This enzyme specifically produced CBCA when expressed in the yeast Pichia pastoris that was fed with CBGA and the disclosed CBCAS nucleotide sequence shared a 96% DNA sequence identity with THCAS. Further whole-genome sequencing studies demonstrated multiple tandem copies of CBCAS and CBCAS-like sequences with over 99% DNA sequence identity to each other in genomic “cassettes” in most, but not all high-THC and high-CBD cannabis sequenced (McKernan et al., 2020) (Grassa et al., 2018) (Laverty et al., 2019). Despite their apparent abundance in most cannabis genomes, transcript expression (e.g. messenger RNA) of these CBCAS or CBCAS-like genes in cannabis tissue has never been shown. Indeed, recent in-depth transcriptome sequencing of type I and type II cannabis flower did not find any sign of full or partial-length CBCAS transcripts, although trace amounts of CBC were detected (McGarvey et al., 2020).

The present technology stems from the recognition, as disclosed herein, of a novel expression-altering variant CBCAS allele, the transcript of which is expressed in the glandular trichomes of a cannabis plant with increased levels of CBCA and/or CBC. This is the first demonstration of an expressed CBCAS allele. The present technology recognizes that presence and the expression of this expression-altering variant CBCAS allele can provide insight regarding accumulation of cannabichromenic acid (CBCA) and/or cannabichromene (CBC) in a Cannabis plant.

Thus, the current technology provides a CBCA-enriched Cannabis plant from a high-cannabinoid producing background which has more immediate value and requires less breeding to stabilize.

The nucleic acid sequence of the expression-altering variant CBCA synthase gene disclosed herein is provided in SEQ ID NO: 8.

The amino acid sequence encoded by the expression-altering variant CBCA synthase allele is provided in SEQ ID NO: 9.

In some embodiments, the present technology relates to an expression-altering variant CBCAS allele. In some implementations of these embodiments, the expression-altering variant CBCAS allele causes the expression and/or an increase in expression of the CBCAS gene and of the associated gene product as compared to the non-expression-altering variant CBCAS allele.

In some implementations of these embodiments, the expression-altering variant CBCAS allele comprises an expression-altering variation that causes expression or an increase in expression of an otherwise not expressed or minimally expressed CBCAS allele.

In some further implementations of these embodiments, the expression-altering variant CBCAS allele comprises an expression-altering variation that causes production and/or an increase in production of the associated gene product compared to the wild type associated gene product.

In some implementations of these embodiments, the expression-altering variation of the expression-altering variant CBCAS allele comprises one or more SNP.

In some implementations of these embodiments, the one or more SNP is in the region that encodes for amino acids spanning between position 10 and 20 of the corresponding polypeptide.

In some further implementations the expression-altering variant CBCAS allele comprises a SNP at position 45. In some instances, the SNP is A45G.

In some further implementations the expression-altering variant CBCAS allele comprises a SNP at position 300. In some instances, the SNP is C300T.

In some further implementations the expression-altering variant CBCAS allele comprises a SNP at position 45 and a SNP at position 300. In some instances, the SNP at position 45 is A45G and the SNP at position 300 is C300T.

In some embodiments, the present technology relates to an expression-altering variant CBCA synthase allele. In some implementations of these embodiments, the expression-altering variant CBCAS allele causes production of the associated gene product which under non-expression-altering variant CBCAS condition is not produced or is minimally produced.

In some implementations of these embodiments, the expression-altering variant CBCAS allele comprises an expression-altering variation that causes production or an increase in production of the associated gene product.

In some implementations of these embodiments, the expression-altering variation of the expression-altering variant CBCAS allele comprises a SNP in the region that encodes for amino acids spanning between position 10 and 20 of the corresponding polypeptide.

In some further implementations the expression-altering variant CBCAS allele comprises a SNP at position 45. In some instances, the SNP is A45G. In some further implementations, the expression-altering variant result in an amino acid change at position 15. In some instance, the amino acid change is I15M.

In some instances, the SNP at position 45, may or may not be involved in the alteration of expression of the CBCAS allele.

In some instances, the SNP at position 300, may or may not be involved in the alteration of expression of the CBCAS allele.

In some instances, the expression-altering variant CBCAS allele promotes conversion of CBGA to CBCA leading to a Cannabis plant with a CBCA-enriched cannabinoid fraction and/or a CBC-enriched cannabinoid fraction.

In some instances, the expression-altering variant CBCAS allele promotes conversion of CBGA to CBCA leading to a CBCA-enriched Cannabis plant from a high-cannabinoid producing background.

In some embodiments, the present technology relates to an isolated nucleic acid molecule having at least about 75%, or at least about 80%, or at least about 85%, at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity to SEQ ID NO: 8, while conserving the expression-altering variation of the present disclosure that causes expression of the CBCAS of the present technology.

In some embodiments, the present technology relates to nucleic acid molecules that hybridize to the above disclosed sequences. Hybridization conditions may be stringent in that hybridization will occur if there is at least about a 96% or about a 97% sequence identity with the nucleic acid molecule in SEQ ID NO: 8. The stringent conditions may include those used for known Southern hybridizations such as, for example, incubation overnight at 42° C. in a solution having 50% formamide, 5×SSC (150 mM NaCl, 15 mM trisodium citrate), 50 mM sodium phosphate (pH 7.6), 5×Denhardt's solution, 10% dextran sulfate, and 20 mg/mL denatured, sheared salmon sperm DNA, following by washing the hybridization support in 0.1×SSC at about 65° C. Other known hybridization conditions are well known and are described in Sambrook et al., Molecular Cloning: A Laboratory Manual, Third Edition, Cold Spring Harbor, N.Y. (2001).

In some embodiments, the isolated nucleic acid molecules of the present technology comprise a nucleic acid sequence as set forth in SEQ ID NO: 8 or a fragment thereof, wherein the fragment therefor conserves the expression-altering variation of the present disclosure.

Fragments contemplated by the present technology, but not limited to, include nucleic acid molecules having a nucleic acid sequence as set forth in SEQ ID NO: 1, 2, 3, 4, 5, 7, 8, 12, 13, 16, 17, 18 and 19 as well as sequences with at least or about 85% or more sequence identity thereto are also contemplated.

In some embodiments, the isolated nucleic acid molecule of the present technology comprises at least and/or up to or about 15, at least and/or up to or about 20 at least and/or up to or about 25, at least and/or up to or about 30, at least and/or up to or about 40 at least and/or up to or about 50, at least and/or up to or about 60, at least and/or up to or about 70, at least and/or up to or about 80, at least and/or up to or about 90, at least and/or up to 100, at least or up to or about 200, at least or up to or about 300, at least or up or about 400, at least or up to or about 500, at least or up to or about 600, at least or up to or about 700, at least or up to or about 800, at least or up to or about 900, at least or up to or about 1000, at least or up to or about 1100, at least or up to or about 1200, at least or up to or about 1300, at least or up to or about 1400 or at least or up to or about 1500 or about 1600 contiguous nucleotides of SEQ ID NO: 8. For example, the nucleic acid molecule can be from 15 contiguous nucleotides up to 1638 contiguous nucleotides or any range or number of nucleotides there between.

The length of the nucleic acid molecule described above will depend on the intended use. For example, if the intended use is as a primer or probe, for example, for PCR amplification or for screening a library, the length of the nucleic acid molecule will be less than the full length sequence, such as a fragment of for example, about 15 to about 50 nucleotides, or at least about 15 nucleotides of SEQ ID NO: 8 and/or its complement. In these embodiments, the primers or probes may be substantially identical to a highly conserved region of the nucleic acid sequence or may be substantially identical to either the 5′ or 3′ end of the DNA sequence. In some cases, these primers or probes may use universal bases in some positions so as to be ‘substantially identical’ but still provide flexibility in sequence recognition. Suitable primer and probe hybridization conditions are well known in the art.

In an embodiment, the nucleic acid molecule can be used as a primer and for example comprises the nucleic acid sequence as set forth in any one of SEQ ID NO: 1, 2, 3, 4, 5, 6, 7, 8, 12, 13, 16, 17, 18 and 19.

In an embodiment, the nucleic acid is conjugated to and/or comprises a heterologous moiety, such as a unique tail, purification tag or detectable label. The unique tail can be a specific nucleic acid sequence. The nucleic acid can for example be end labelled (5′ or 3′) or the label can be incorporated randomly during synthesis.

In one embodiment, the present technology provides an isolated nucleic acid that encodes for the polypeptide having an amino acid sequence as set forth in SEQ ID NO: 9 or a fragment thereof.

In some embodiments, the present technology relates to an isolated polypeptide having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% identity to the amino acid sequence as set forth in SEQ ID NO: 9. In some implementations of these embodiments, the isolated polypeptide comprises the expression-altering variation that results in production of CBCAS that promotes conversion of CBGA to CBCA.

In some embodiments, the present technology relates to an antibody that specifically binds a polypeptide as set forth in SEQ ID NO: 9 or to fragments thereof. In some instances, the antibody is a purified antibody. By “purified” is meant that a given antibody or fragment thereof, whether one that has been removed from nature (isolated from blood serum) or synthesized (produced by recombinant means), has been increased in purity, wherein “purity” is a relative term, not “absolute purity”. In particular aspects, a purified antibody is 60% free, preferably at least about 75% free, and more preferably at least about 90% free from other components with which it is naturally associated or associated following synthesis.

In some embodiments, the present technology relates a construct or an in vitro expression system having an isolated nucleic acid molecule having at least, greater than or about 75% sequence identity to SEQ ID NO: 8. Accordingly, the present technology further relates to a method for preparing a construct or in vitro expression system including such a sequence, or a fragment thereof, for introduction of the sequence or partial sequence in a sense or anti-sense orientation, or a complement thereof, into a cell.

In some embodiments, an extract of the recombinant organism described herein or of a part thereof, such as a recombinant plant extract, comprises an increased level of CBCA and/or CBC. In some embodiments, an extract of the recombinant organism described herein or of a part thereof, such as a recombinant plant extract, comprises an increased level of CBCA and/or CBC in a high-cannabinoid producing background. Accordingly, an aspect of these embodiments includes a cannabinoid or a composition comprising CBCA and/or CBC, produced according to a method or system described herein.

Is some embodiments, the present technology relates to a recombinant organism, host cell or germ tissue (e.g. seed) of the organism comprising a nucleic acid molecule having at least 15 contiguous nucleotides of SEQ ID NO: 8 and/or a construct comprising said isolated and/or purified nucleic acid molecule. In some instances of these embodiments, the at least 15 contiguous nucleotides of SEQ ID NO: 8 include the expression-altering variation that results in expression or an increase in expression of CBCAS promoting conversion of CBDA to CBCA.

In an embodiment, the recombinant organism, cell and/or germ tissue expresses a polypeptide having at least and/or up to about 150, about 175, about 200, about 225, or about 250 amino acids of the polypeptide sequence and optionally at least about 90% sequence identity to as set forth in SEQ ID NO: 9, which conserving the expression-altering variation that results in expression or an increase in expression of CBCAS promoting conversion of CBDA to CBCA.

In some embodiments, the recombinant organism of the present technology is a Cannabis plant that has trichomes with a stalked shape. As used herein, the expression “stalked trichomes” refers to trichomes that are shaped like mushrooms with a bulb at the head of the stalk. In some embodiments, the Cannabis plant of the present technology does not have trichomes that are specific to a prolonged juvenile characteristic.

The recombinant expression vectors of the present technology may also contain nucleic acid sequences which encode a heterologous polypeptide (e.g. fusion moiety) producing a fusion polypeptide when a nucleic acid of interest encoding a polypeptide is introduced into the vector in frame. The heterologous polypeptide can provide for increased expression of the recombinant protein; increased solubility of the recombinant protein; and/or aid in the purification of the target recombinant protein by acting as a ligand in affinity purification. For example, a proteolytic cleavage site may be added between the target recombinant protein to allow separation of the recombinant protein from the fusion moiety subsequent to purification of the fusion polypeptide. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Preferably, the recombinant organism is a recombinant plant, recombinant multicellular microorganism or recombinant insect. Plants are preferably of the genus Cannabis. Microorganisms are preferably bacteria (e.g. Escherichia coli) or yeast (e.g. Saccharomyces cerevisiae, Pichia pastoris). Microorganisms that are unicellular can be considered organisms or cells, including host cells. Insect is preferably Spodoptera frugiperda.

In some embodiments, the present technology also provides for organisms, tissues or cells such as Cannabis plants, Cannabis tissue and Cannabis cells having an expression-altering variant CBCAS expressing CBCAS which promotes conversion of CBGA to CBCA.

In some embodiments, the present technology also provides for organisms, tissues or cells such as Cannabis plants, Cannabis tissue and Cannabis cells having an expression-altering variant CBCAS expressing CBCAS which promotes conversion of CBGA to CBCA and causes an increase in CBCA and/or CBC in the organisms, tissues or cells. In some implementations of these embodiments, the expression of CBCAS promotes conversion of CBGA to CBCA and causes an increase in CBCA and/or CBC in a high-producing cannabinoid organisms, tissues or cells

In some embodiments, the present technology also provides for organisms, tissues or cells that comprise the nucleic acids and/or the polypeptides as defined herein. In some embodiments, the organisms, tissues or cells are plants, plant tissues or plant cells that exhibit CBCAS activity. In some instances, such plants are Cannabis plants and such plant tissues and plant cells are Cannabis tissue and Cannabis cells.

Plants, and in particular Cannabis plants, containing the CBCAS nucleotide sequences of the present technology may be created via known plant transformation methods for example Agrobacterium-mediated transformation, transformation via particle bombardment, pollen tube or protoplast transformation. In these methodological approaches, the gene of interest is incorporated into the genome of the target organism. For example, tissue culture and Agrobacterium mediated transformation of hemp is described in Feeney and Punja, 2003.

Recombinant expression vectors can be introduced into host cells to produce a transformed host cell. Prokaryotic and/or eukaryotic cells can be transformed with nucleic acid by, for example, electroporation or calcium chloride-mediated transformation. For example, nucleic acid can be introduced into mammalian cells via conventional techniques such as calcium phosphate or calcium chloride co-precipitation, DEAE-dextran mediated transfection, lipofectin, viral mediated methods, electroporation or microinjection. Suitable methods for transforming and transfecting cells can be found in Sambrook et al. (Molecular Cloning: A Laboratory Manual, 3rd Edition, Cold Spring Harbor Laboratory Press, 2001), and other laboratory textbooks.

Suitable host cells include a wide variety of eukaryotic cells and prokaryotic cells. For example, the nucleic acids and proteins of the disclosure may be expressed in plant cells, yeast cells or mammalian cells. Plant cells are preferably of the genus Cannabis. Microorganisms are preferably bacteria (e.g. Escherichia coli) or yeast (e.g. Saccharomyces cerevisiae, Pichia pastoris).

Other suitable host cells can be found in Goeddel, Gene Expression Technology: Methods in Enzymology 185, Academic Press, San Diego, Calif (1991). In addition, the proteins of the disclosure may be expressed in prokaryotic cells, such as Escherichia coli (Zhang et al., Science 303(5656): 371-3 (2004)). In addition, a Pseudomonas-based expression system such as Pseudomonas fluorescens can be used (US Patent Application Publication No. US 2005/0186666).

In some embodiments, the present technology also relates to recombinant cells comprising a nucleic acid molecule or polynucleotide of the disclosure. In an embodiment, the nucleic acid molecule results in an increased level of CBCA and/or CBC in the recombinant cell.

Recombinant organisms, cells and tissues described herein may have altered levels of cannabinoid compounds and in particular may have altered levels of CBCA and/or CBC. Expression of the nucleic acid and amino acids sequences of the present technology will result in expression of CBCAS enzyme which may result in increase conversion of CBGA to CBCA and in turn may result in an increased production of CBCA and/or CBC. Expression of the nucleic acid and amino acids sequences of the present technology will result in expression of CBCAS enzyme which may result in increase conversion of CBGA to CBCA and in turn may result in an increased production of CBCA and/or CBC in a high-producing cannabinoid background.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in increased levels of CBCA and/or CBC, for example, about 1-500000%, about 1-25000%, about 1-10000%, about 1-5000%, about 1-2000%, about 1-1000%, about 1-500%, about 1-250%, about 1-100%, about 1-about 50%, about 2-about 50%, about 5-bout 50%, about 10-about 50%, about 25-about 50%, or about 1-about 25% (w/w). In some instances, the control is of the same variety. In some instances, the expression-altered CBCAS enzyme is expressed in the glandular trichomes.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in increased levels of CBCA and/or CBC, by at least about 1.5 times, at least about 2 times, at least about 5 times, at least about 10 times, at least about 11 times, at least about 12 times, at least about 13 times, at least about 14 times, at least about 15 times, at least about 20 times, at least about 25 times, at least about 50 times, or at least about 75 times, or about 100 times, or about 250 times, or about 500 times or about 1000 times, or about 2000 times, or greater than about 2000 times. In some instances, the expression-altered CBCAS enzyme is expressed in the glandular trichomes.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in CBDA/CBCA ratio of anywhere between about 15:1 and about 1:2000. In some instances, the expression-altered CBCAS enzyme is expressed in the glandular trichomes.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in total CBD/CBC ratio of anywhere between about 15:1 and about 1:2000.

In some embodiments, expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in total CBD/CBC ratio of between about 15:1 and about 1:1, or between about 12:1 and about 1:1, or between about 10:1 and about 1:1, or between about 9:1 and about 1:1, or between about 8:1 and about 1:1, or between about 7:1 and about 1:1, or between about 6:1 and about 1:1, or between about 5:1 and about 1:1, or between about 4:1 and about 1:1, or between about 3:1 and about 1:1, or between about 2:1 and about 1:1.

In some embodiments, expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in total CBD/CBC ratio of between about 1:1 and about 1:2000, or between about 1:1 and about 1:1000, or between about 1:1 and about 1:500, or between about 1:1 and about 1:250, or between about 1:1 and about 1:100, or between about 1:1 and about 1:50, or between about 1:1 and about 1:25, or between about 1:1 and about 1:20, or between about 1:1 and about 1:15, or between about 1:1 and about 1:10, or between about 1:1 and about 1:5.

In some instances, the expression-altered CBCAS enzyme is expressed in the glandular trichomes. In some instances, CBD includes CBDA and CBC includes CBCA.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total CBCA/CBC content at maturity of at least about 1%, at least about 2%, at least about 3%, at least about 2%, at least about 5% at least about 10%, at least about 15%, or at least about 20% based on dry weight of total material. In some instances, the expression-altered CBCAS enzyme is expressed in the glandular trichomes. In some instances, the dry weight of total material is dry flower weight.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total cannabinoid content at maturity of at least about 3%, at least about 5%, at least about 10%, at least about 15%, at least about 20% at least about 25%, at least about 30%, or at least about 35% based on total weight of cannabinoids In some instances, the expression-altered CBCAS enzyme is expressed in the glandular trichomes. In some instances, the dry weight of total material is dry flower weight.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total CBCA/CBC content at maturity of at least about 1%, at least about 2%, at least about 3%, at least about 2%, at least about 5% at least about 10%, at least about 15%, or at least 20% based on dry weight of total material; and a total cannabinoid content at maturity of at least about 3%, at least about 5%, at least about 10%, at least about 15%, at least about 20% at least about 25%, or at least about 30%, or at least 35% based on total weight of cannabinoids In some instances, the expression-altered CBCAS enzyme is expressed in the glandular trichomes. In some instances, the dry weight of total material is dry flower weight.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total CBCA/CBC content at maturity of at least about 1%, at least about 2%, at least about 3%, at least about 2%, at least about 5% at least about 10%, at least about 15% based on dry weight of total material; and a total cannabinoid content at maturity of at least about 3%, at least about 5%, at least about 10%, at least about 15%, at least about 20% at least about 25%, or at least about 30%, based on total weight of cannabinoids. In some instances, the expression-altered CBCAS enzyme is expressed in the glandular trichomes. In some instances, the dry weight of total material is dry flower weight.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total CBCA/CBC content at maturity of at least about 1% based on dry weight of total material and a total cannabinoid content at maturity of at least about 3%. In some instances, the expression-altered CBCAS enzyme is expressed in the glandular trichomes. In some instances, the dry weight of total material is dry flower weight.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total CBCA/CBC content at maturity of at least about 2% based on dry weight of total material and a total cannabinoid content at maturity of at least about 3%. In some instances, the expression-altered CBCAS enzyme is expressed in the glandular trichomes. In some instances, the dry weight of total material is dry flower weight.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total CBCA/CBC content at maturity of at least about 3% based on dry weight of total material and a total cannabinoid content at maturity of at least about 10%. In some instances, the expression-altered CBCAS enzyme is expressed in the glandular trichomes. In some instances, the dry weight of total material is dry flower weight.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total CBCA/CBC content at maturity of at least about 4% based on dry weight of total material and a total cannabinoid content at maturity of at least about 10%. In some instances, the expression-altered CBCAS enzyme is expressed in the glandular trichomes. In some instances, the dry weight of total material is dry flower weight.

Expression of the expression-altered CBCAS enzyme of the present disclosure in a cell, tissue or plant compared to a control cell, tissue or plant that either does not express the expression-altered CBCAS enzyme or that minimally expresses the expression-altered CBCAS enzyme will result in a total CBCA/CBC content at maturity of at least about 5% based on dry weight of total material and a total cannabinoid content at maturity of at least about 10%. In some instances, the expression-altered CBCAS enzyme is expressed on the glandular trichomes. In some instances, the dry weight of total material is dry flower weight.

In some instances, the control is of the same plant variety.

In Cannabis plants the transmission of the expression-altering variation defined herein and the production or enhanced production of CBCA and/or CBC could be achieved through breeding and selection as well as genetic engineering with the use of genes encoding the enzymes of cannabinoid biosynthetic pathways, e.g. the CBCAS gene in this disclosure.

In some embodiments, the present technology relates to methods of altering levels of CBCA and/or CBC compounds in an organism, cell or tissue, said method comprising using a nucleic acid molecule of the present disclosure or a fragment thereof to cause an expression-altered CBCAS to be expressed in the organism, cell or tissue. In some implementations of these embodiments, the levels of CBCA and/or CBC compounds is increased by making the recombinant cells expressing the expression-altering variant CBCAS of the present technology.

In one embodiment, the present technology relates to methods for increasing the production of CBCA and/or CBC in cells of an organism. In some implementations of this embodiment, the method comprises introducing into the cells of the organism, a vector comprising a nucleic acid comprising SEQ ID NO: 8 or a fragment thereof conserving the expression-altering variation as disclosed herein. The vector comprises a nucleic acid having at least or about 75% sequence identity to SEQ ID NO: 8 while retaining the expression-altering variation to produce recombinant cells. The method may further comprise the step of culturing and/or growing the recombinant cells under conditions that permit expression of the nucleic acid; and optionally isolating and/or purifying CBCA and/or CBC. The recombinant cell can be transiently expressing, inducibly expressing and/or stably expressing.

In some embodiments, a Cannabis plant genome to be modified by the methods of the present technology includes a CBCA synthase gene sequence. In some instances, a Cannabis plant genome to be modified by the methods of the present technology includes a wild-type CBCA synthase gene sequence. In some instances, the Cannabis plant genome is homozygous for a wild-type CBCA synthase gene sequence or is heterozygous for a wild-type CBCA synthase gene sequence, or is homozygous for an expression-altering variant CBCA synthase gene sequence. In some embodiments, a Cannabis plant genome is heterozygous for an expression-altering variant CBCA synthase gene sequence. In some embodiments, a Cannabis plant genome includes a CBCA synthase gene sequence that is or comprises a sequence that is 70%, 75%, 80%, 85%, 85%, 87%, 88%, 89%, 90%, 91%, 92%, 93%, 94%, 95%, 96%, 97%, 98%, 99%, or 100% identical to SEQ ID NO: 8, or a portion thereof.

The recombinant expression vector of the present technology, in addition to containing a nucleic acid molecule or polynucleotide disclosed herein, may contain regulatory sequences for the transcription and translation of the inserted nucleic acid molecule. Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. As will also be apparent to persons skilled in the art, and as described elsewhere (Meyer, 1995; Datla et al., 1997), it is possible to utilize promoters to direct any intended up- or down-regulation of transgene expression using constitutive promoters (e.g., those based on CaMV35S), or by using promoters which can target gene expression to particular cells, tissues (e.g., napin promoter for expression of transgenes in developing seed cotyledons), organs (e.g., roots, leaves), to a particular developmental stage, or in response to a particular external stimulus (e.g., heat shock).

In some embodiments, the present technology relates to a method for detecting the presence of a CBCAS gene sequence or a portion thereof that comprises the expression-altering variation of the present disclosure. The method includes amplifying a CBCAS gene sequence or portion thereof comprising the expression-altering variation from a sample that comprises nucleic acid from a Cannabis plant. The amplification of a CBCAS gene sequence includes contacting nucleic acid from a Cannabis plant with a forward CBCAS primer that is complementary to a sequence that is 200-1000 nucleotides upstream of a CBCAS open reading frame. In some embodiments, a forward CBCAS primer is complementary to a sequence that is 200 to 800 nucleotides, 200 to 600 nucleotides, 200 to 400 nucleotides upstream of a CBCAS open reading frame. In some certain embodiments, a forward CBCAS primer is complementary to a sequence that is at least 70%, 75%, 80%, 85%, 85%, 87%, 88%, 89%, 90%, 91%, 92%, 93%, 94%, 95%, 96%, 97%, 98%, 99%, or 100% identical to a portion of SEQ ID NO: 8. In some certain embodiments, a forward CBCAS primer is at 20 to 60 nucleotides long. The amplification of a CBCAS gene sequence includes contacting nucleic acid from a Cannabis plant with a reverse CBCAS primer that is complementary to a sequence that is 200-1000 nucleotides downstream of a CBCAS open reading frame. In some embodiments, a reverse CBCAS primer is complementary to a sequence that is 200 to 800 nucleotides, 200 to 600 nucleotides, 200 to 400 nucleotides downstream of a CBCAS open reading frame. In some certain embodiments, a reverse CBCAS primer is complementary to a sequence that is at least 70%, 75%, 80%, 85%, 85%, 87%, 88%, 89%, 90%, 91%, 92%, 93%, 94%, 95%, 96%, 97%, 98%, 99%, or 100% identical to a portion of SEQ ID NO: 8. In some certain embodiments, a reverse CBCAS primer is about 20 to 60 nucleotides long.

In some embodiments, a method of the present disclosure includes detecting the presence of a polymorphism within a CBCAS gene sequence in a sample that comprises nucleic acid from the Cannabis plant. In some embodiments, a polymorphism within a CBCAS gene sequence results in a polypeptide that comprises an amino acid change between position 10 and 20 of the amino acid sequence.

In some embodiments, the present technology relates to a method for controlling the conversion of CBGA to CBCA in Cannabis. In some implementations, the method comprises obtaining an endonuclease enzyme which targets a nucleic acid sequence coding for CBCAS and introducing the endonuclease enzyme into the genome of a plant of genus Cannabis. In some implementations of these embodiments, the endonuclease enzyme is made in vitro. The introduction of the endonuclease enzyme may be accomplished through inoculating the plant with a bacteria comprising a genetic sequence for an endonuclease enzyme. Once inoculated, the bacteria make plant cells which will then produce the endonuclease enzyme. In some instances, inoculating comprises placing the Cannabis plant in a vacuum chamber with a bacterial solution comprising the endonuclease enzyme and removing air drawing the bacterial solution comprising the endonuclease enzyme into the plan. In some instances, the inoculation comprises spraying the Cannabis plant with an endonuclease enzyme. In some instances, spraying is accomplished using biolistic particles bombardment.

In some implementations of these embodiments, the endonuclease enzyme is a CRISPR/Cas9 system. As used herein, the term “CRISPR” refers to an acronym that means Clustered Regularly Interspaced Short Palindromic Repeats of DNA sequences. CRISPR is a series of repeated DNA sequences with unique DNA sequences in between the repeats. RNA transcribed from the unique strands of DNA serves as guides for directing cleaving. CRISPR is used as a gene editing tool. In one embodiment, CRISPR is used in conjunction with a Cas9 protein. As used herein, the term “Cas” refers to CRISPR associated proteins that act as enzymes cutting the genome at specific sequences. Cas9 refers to a specific group of proteins known in the art. RNA sequences made from CRISPR direct Cas9 enzymes to cut certain sequences found in the genome. Other classes of Cas are also acceptable. In some instances, the CRISPR/Cas9 system cleaves one or two chromosomal strands at known Cas9 protein domains. In one embodiment, one of the two chromosomal strands is mutated. In one embodiment, two of the two chromosomal strands are mutated. As used herein, the term “chromosomal strand” refers to a sequence of DNA within the chromosome. When the CRISPR/Cas9 system cleaves the chromosomal strands, the strands are cut leaving the possibility of one or two strands being mutated, either the template strand or coding strand. The CRISPR/Cas9 system cleaves both strands inducing non-homologous end joining (NHEJ) and then an insertion of a DNA sequence that includes the activity-altering change thereby causing the encoded protein to mutate and become minimally functional or non-functional. In one embodiment, the CRISPR/Cas9 system cleaves both strands causing homology directed repair (HDR) to occur. In some instances, a donor DNA strand is inserted into the space between the cleaved strands preventing random mutation. In one embodiment, the donor DNA strand is a DNA sequence coding for an expression-altering variant CBCAS enzyme.

In some embodiments, the expression-altering variation results in expression or an increase in expression of a CBCAS enzyme.

In one embodiment, the methods disclosed herein comprise a RNA guide. As used herein, the term “RNA guide” refers to a strand of RNA recognizing a specific sequence of genetic material and directing where the endonuclease enzyme to cut. In one embodiment, the RNA guide directs the endonuclease enzyme to cleave chromosomal strands coding for a cannabinoid synthesis enzyme. In one embodiment, the RNA guide directs the CRISPR/Cas9 system to cleave chromosomal strands coding for a cannabinoids synthesis enzyme. In one embodiment, the RNA guide directs the CRISPR/Cas9 system to target a THCAS expression gene. Within the context of this disclosure, other examples of endonuclease enzymes include SpCas9 from Strptococcus pyrogenes and others. Additionally, SpCas9 have differing Protospacer Adjacent Motif (PAM) sequences from NGG, which may offer other advantages. In one example, a SpCas9 has a smaller coding sequence. Other examples of proteins that work with CRISPRs or RNA guides include Cpf1, which can be used for cutting DNA strands with overhanging ends instead of blunt ends, or C2c2 for cutting RNA with an RNA guide. As used herein, the term “PAM” refers to a short DNA base pair sequence immediately following the DNA sequence targeted by an endonuclease enzyme.

In one embodiment, the methods disclosed herein comprise an endonuclease enzyme and an RNA guide. In one embodiment, the methods disclosed herein comprise a guide RNA transcribed in vitro. In one embodiment, the methods disclosed herein comprise a guide RNA transcribed in vivo.

In one embodiment, the methods disclosed herein comprise introducing a Cas9 enzyme and guide RNA expression cassette into the genome.

In some embodiments, the present disclosure provides kits comprising materials useful for amplification and detection and/or sequencing of Cannabis plant nucleic acid (e.g., DNA). In some embodiments, Cannabis plant nucleic acid sample includes detection of all or part of a CBCAS gene sequence as described herein. In some embodiments, a kit in accordance of the present disclosure is portable.

Suitable amplification reaction reagents that can be included in an inventive kit include, for example, one or more of: buffers; enzymes having polymerase activity; enzyme cofactors such as magnesium or manganese; salts; nicotinamide adenide dinuclease (NAD); and deoxynucleoside triphosphates (dNTPs) such as, for example, deoxyadenosine triphospate; deoxyguanosine triphosphate, deoxycytidine triphosphate and deoxythymidine triphosphate, biotinylated dNTPs, suitable for carrying out the amplification reactions.

In some embodiments, a kit comprises 1, 2, 3, 4, 5, 6, 7, 8, 9, 10, 11, 12, 13, 14, 15, 16, 17, 18, 19, 20, or more primer sequences for in vitro nucleic acid amplification. Primer sequences may be suitable for in vitro nucleic acid amplification with any of the methods described herein (e.g., QT-PCR, LAMP, etc.). In some embodiments, a kit of the present disclosure includes reagents suitable to perform a colorimetric LAMP assay for amplification of one or more Cannabis gene sequences as described herein.

Depending on the procedure, a kit may further include one or more of: wash buffers and/or reagents, hybridization buffers and/or reagents, labeling buffers and/or reagents, and detection means. The buffers and/or reagents included in a kit are preferably optimized for the particular amplification/detection technique for which a kit is intended. Protocols for using these buffers and reagents for performing different steps of the procedure may also be included in a kit.

In some embodiments, a kit may further include one or more reagents for preparation of nucleic acid from a plant sample. For example, a kit may further include one or more of a lysis buffer, a DNA preparation solution (e.g., a solution for extraction and/or purification of DNA). Kits may also contain reagents for the isolation of nucleic acids from biological specimen prior to amplification. Protocols for using these reagents for performing different steps of the procedure may also be included in a kit.

Furthermore, kits may be provided with an internal control as a check on the amplification procedure and to prevent occurrence of false negative test results due to failures in the amplification procedure. An optimal control sequence is selected in such a way that it will not compete with the target nucleic acid sequence in the amplification reaction (as described above).

In some embodiments, a kit may further include reagents for an amplification assay to characterize the gender of a Cannabis plant.

Reagents may be supplied in a solid (e.g., lyophilized) or liquid form. Kits of the present disclosure may optionally comprise different containers (e.g., vial, ampoule, test tube, flask or bottle) for each individual buffer and/or reagent. In some embodiments, each component will generally be suitable as aliquoted in its respective container or provided in a concentrated form. Other containers suitable for conducting certain steps of inventive amplification/detection assay(s) may also be provided. Individual containers of a kit are preferably maintained in close confinement for commercial sale.

A kit may also comprise instructions for using the amplification reaction reagents, primer sets, primer/probe sets according to the present disclosure. Instructions for using a kit according to one or more methods of the present disclosure may comprise instructions for processing the biological sample, extracting nucleic acid molecules, and/or performing one or more amplification reactions; and/or instructions for interpreting results.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Identification of a CBC-Enriched Cannabis Plant

A high-CBD hemp breeding program was established to develop novel strains. For all plant experiments described herein vegetative mother plants for each strain were first established by meristem explant cloning (with 2-4 expanded leaves) and rooted in rockwool cubes (Grodan®) under fluorescent lighting. Once clones had developed roots through the rockwool cubes they were transferred into 3-gallon deep water culture set ups with constant aeration from an aquarium pump and diffusion stone. Plants were grown for two to four weeks under Ray44 88W lights (Fluence®) under 18-hour light/6 hour dark cycle before being transferred to a reflective growing chamber with Viparspectra 600W (Viparspectra®) lights to initiate flowering under 12 hour light/12 hour dark cycle. Hydroponic vegetative stage nutrients Foliage pro 9:3:6 (Dyna-Gro®) were used at 1-2 tsp/gallon with antimicrobial Clear Rez (EZClone®) being supplemented at ˜10 ml/gallon and then pH adjusted to 5.8. Flowering stage nutrients Bloom 3:12:6 (Dyna-Gro®) were used at 1-2 tsp/gallon with antimicrobial Clear Rez (EZClone®) being supplemented at ˜10 ml/gallon and then pH adjusted to 6. Temperature was maintained at around 22° C. and relative humidity was around 30%.

Cannabinoid content was assessed via high performance liquid chromatography (HPLC) from mature flower samples that had been pollenated and had set seed, at 12 weeks post flower initiation. For all HPLC analysis presented herein, harvested flower was dried on a rack at around 30% humidity, ambient temperature, for one week. Approximately 1 g of flower sample was ground and weighed and solvated into 10 mL of ethanol within a glass scintillation vial and sonicated for 30 minutes. The sample solution was filtered through a 0.22 μm nylon filter and then diluted 10 and 200-fold for HPLC analysis in 80/20 acetonitrile/isopropyl alcohol. Separation of 13 cannabinoids was achieved using a Raptor ARC-C18, 2.7 μm, 150×4.6 mm (Restek®) column with a gradient mobile phase consisting of A) water with 0.1% formic acid and B) acetonitrile+0.1% formic acid at a constant flow rate of 0.75 mL/min. The mobile phase composition started at 74% B, ramped to 78% B over 6 minutes, then ramped to 86% B between 6.01-10 minutes, held at 95% B from 10.01-11.50 minutes, and then returned to starting conditions at 11.51 (74% B) for 3.5 minutes. The column compartment was held at 4° C. while the autosampler remained at room temperature. 10 μL of each standard and sample was injected. A DAD (diode array detector) was employed for quantification of analytes using the 220 nm signal (no reference) as output. The R2 values of all calibration curves >0.995 and quantification of analytes from the CVS (calibration verification standard injected ˜10 injections) was within 10% the expected value. Total cannabinoid percentages were calculated as % weight neutral cannabinoid+(% weight acid cannabinoid*0.877).

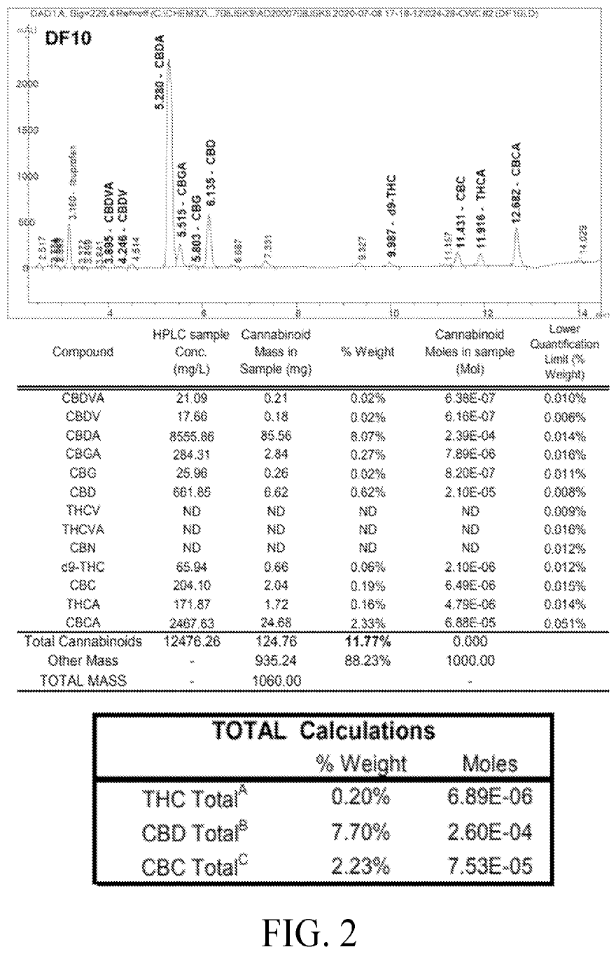

One cultivar (herein referred to as “CGC1”) was isolated that showed a high level of total CBC (CBCA and CBC) content of over 2% by dry weight in de-seeded flower material (FIG. 2).

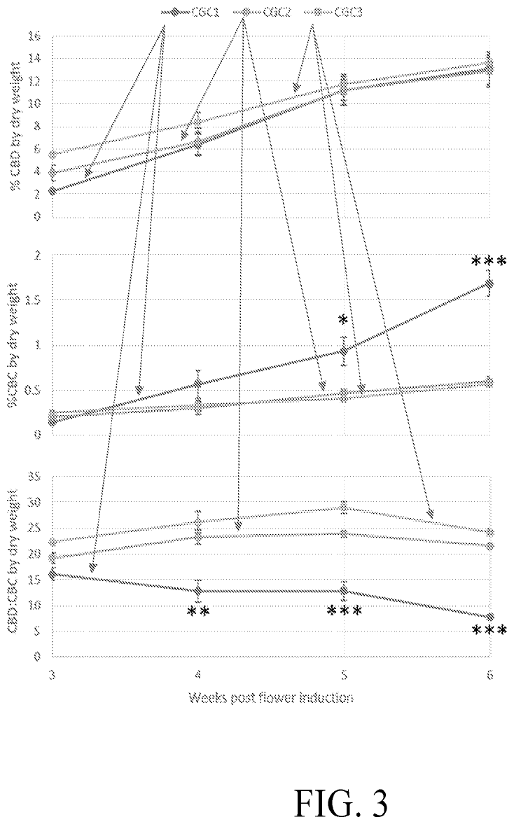

To asses the ability of strain CGC1 to produce elevated levels of CBC/CBCA during flowering in the absence of pollination, 3 or 4 clone plants of strain CGC1 as well as two additional but genetically unique high-CBD strains without enriched CBC/CBCA content, named herein as CGC2 and CGC3, were flowered in parallel and sampled weekly for cannabinoid analysis from weeks 3 through 6 post flower initiation. Total CBD and CBC content and CBD:CBC ratio along the time course analysis are shown in FIG. 3. CBD content in CGC1 did not significantly differ from both CGC2 and CGC3 at any time (see top panel of FIG. 3). However, CBC content in CGC1 was significantly higher than both CGC2 and CGC3 by week 5 (CGC1 0.94±0.16%; CGC2 0.47±0.04%; CGC3 0.41±0.04%) as well as week 6 (CGC1 1.69±0.15%; CGC2 0.60±0.06%; CGC3 0.56±0.01%) where all CGC1 flower samples tested well over 1% total CBC by dry weight (see middle panel of FIG. 3). Further, although there was no significant difference at week 3, the CBD:CBC ratio was significantly lower for CBC1 compared to both CGC2 and CGC3 from week 4 onwards (see bottom panel of FIG. 3). At week 6, the total CBD:CBC ratio for all CGC1 samples was well below 10 but above 20 for all samples from CGC2 and CGC3 (CGC1 7.74±0.37; CGC2 21.54±0.42; CGC3 24.19±0.66). Collectively these data show the unique ability of CGC1 to accumulate elevated CBCA/CBC levels during flowering.

### Example 2—Identification of a Unique CBCAS Allele Expressed in Strain CGC1 Trichomes

To test the possibility that CGC1 accumulated higher levels of CBC compared to CGC2 and CGC3 because of CBCAS expression during flowering not present in strains CGC2 and CGC3, glandular trichomes were isolated and purified from flowers for the three strains at 6 weeks post flower initiation using an established protocol (Braich et al., 2019) with the modifications that trichomes were resuspended in 10 ml of phosphate-buffered saline, passed through 120 μm nylon mesh to remove large non-trichome material, then centrifuged to pellet trichomes at 500×g for 1 min. Supernatant was removed, trichomes were resuspended in 1 ml of lysis buffer and total RNA was isolated using an RNeasy miniprep kit (Qiagen) using manufacturer's instructions. Residual genomic DNA (gDNA) was removed from total RNA using RQ1 RNase-Free DNAse (Promega) using manufacturer's instructions. First-strand complimentary DNA (cDNA) was synthesized from equal amounts of gDNA-free total RNA for each strain using Superscript IV Reverse Transcriptase (Invitrogen) and oligo dT primers using manufacturer's instructions.

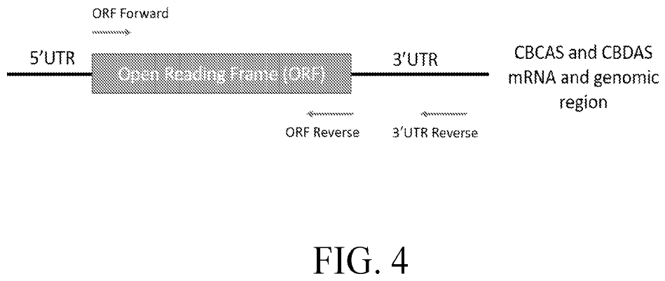

Polymerase chain reaction (PCR) oligonucleotide primers were developed to specifically amplify CBCAS or CBDAS coding regions (FIG. 4). Primers used were as identified in Table 1.

TABLE 1
PCR primers for amplification of CBCAS or CBDAS coding regions

| | |
|---|---|
| CBCAS ORF Forward | 5′-AAATGAATTGCTCAACATTC-3′ (SEQ ID NO: 1) |
| CBCAS ORF Reverse | 5′-GTAGATAATTAATGATGACGCG-3′ (SEQ ID NO: 2) |
| CBCAS 3′UTR Reverse | 5′-GGGAGCATACATAGTATGGG-3′ (SEQ ID NO: 3) |
| CBDAS ORF Forward | 5′-ATGAAGTGCTCAACATTC-3′ (SEQ ID NO: 4) |
| CBDAS ORF Reverse | 5′-TAAGATCATTAATGACGATGCC-3′ (SEQ ID NO: 5) |
| CBDAS 3′UTR Reverse | 5′-ATACACAGTACATCCGGAC-3′ (SEQ ID NO: 6) |

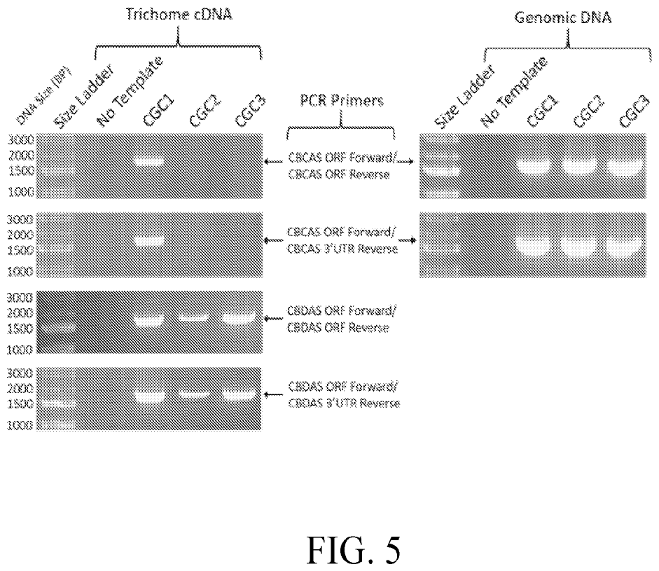

PCR was performed on trichome cDNA template using Q5 enzyme (New England Biolabs®) using manufacturer's instructions with the following thermal cycler program: 1) 98° C. for 30 sec; 2) 98° C. for 10 sec; 3) primer-set specific temperature for 20 sec; 4) 72° C. for 1 min (repeat 2-4 34×); 5) 72° C. for 2 min; and 6) Hold at 4° C. Primer set specific annealing temperatures were determined with manufacturer's web tool (https://tmcalculator.neb.com/#!/main). PCR product was separated on a 1% agarose gel and visualized with SYBR Safe DNA gel stain (ThermoFisher®). A control PCR was used without template DNA. Results showed that CBCAS was only expressed in trichomes of CGC1 but not CGC2 or CGC3 (FIG. 5; left panel). As expected, CBDAS was expressed in the trichomes of all three strains and served as a control for cDNA synthesis from trichome total RNA. This result is the first demonstration of CBCAS transcript expression in cannabis trichomes and suggests that CBCAS expression is correlated with elevated CBC levels observed in CGC1. This data also suggests that CBCAS expression in trichomes is rare amongst Cannabis strains.

To determine if CBCAS was present in the genome of CGC2 and CGC3, but not expressed in trichomes, genomic DNA (gDNA) was isolated from CGC1, CGC2, and CGC3. Young leaf tissue was collected (20 mg) and was homogenized by bead beating. gDNA was extracted using a Maxwell® RSC Plant DNA Kit and Maxwell® RSC instrument (Promega®) using manufacturer's instructions. PCR was performed with gDNA template using CBCAS ORF Forward primer with either CBCAS ORF Reverse primer or CBCAS 3′UTR Reverse primer as described above. Results showed amplification of CBCAS in all three strains (FIG. 5; right panel) suggesting that CBCAS is present in the genome of all three strains but is exclusively expressed in the trichomes of CGC1.

To determine the sequence of the CBCAS allele expressed in CGC1 trichomes, the amplified CBCAS gel bands (FIG. 5; left panel) were excised, DNA was purified and subjected to Sanger sequencing using respective forward or reverse primers as well as a an internal reverse sequencing primer 5′-CTAAAGTGTCCACCTACGCC-3′(SEQ ID NO: 7) for complete sequencing coverage of the CBCAS 5′ end. The full CBCAS sequences were manually curated. Results showed the expression of a single CBCAS nucleic acid molecule named herein as CBCASexpressed and having the nucleic acid sequence as depicted in SEQ ID NO 8.

The amino acid sequence encoded by the nucleic acid sequence of CBCASexpressed is provided in SEQ ID NO: 9.

BLAST analysis of all cannabis nucleotide sequences deposited on the National Center for Biotechnology Information (NCBI) with CBCASexpressed nucleotide sequence as a query found a closest match nucleic acid molecule named herein as CBCASclosestNCBI and having the nucleic acid sequence as depicted in SEQ ID NO 10.

The amino acid sequence encoded by the nucleic acid sequence of CBCASclosestNCBI is provided in SEQ ID NO: 11.

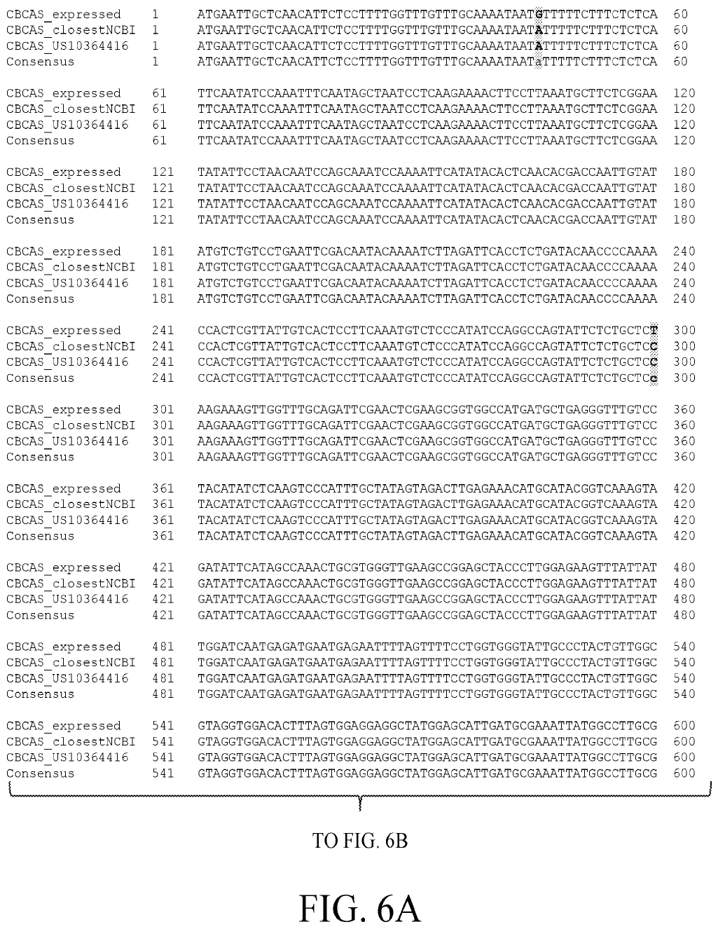

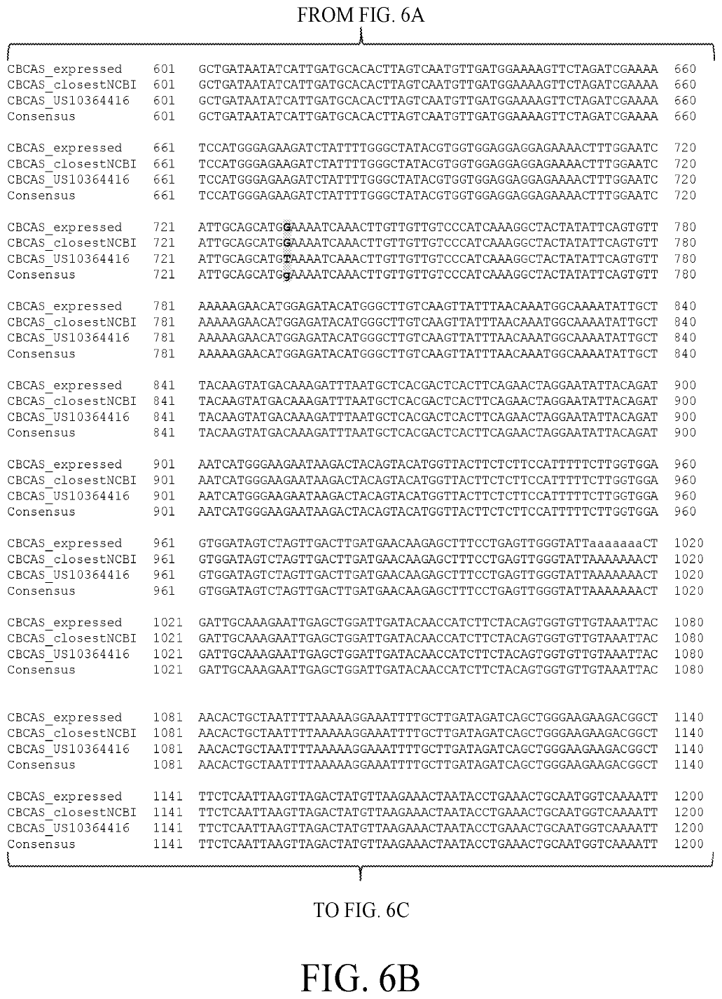

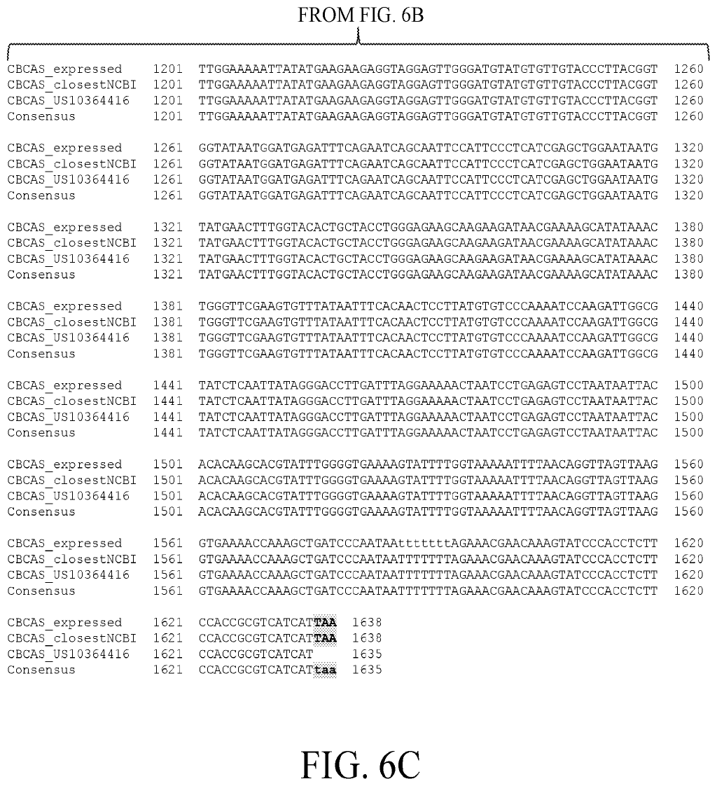

CBCASclosestNCBI was found in the whole genome assembly sequences of multiple strains deposited on NCBI including Type I Purple Kush and Type III hemp Finola (Laverty et al., 2019), Type II Jamaica Lion (McKernan et al., 2020), and others. Nucleotide sequences of CBCASexpressed, CBCASclosestNCBI, and the CBCAS sequence disclosed in U.S. patent Ser. No. 10/364,416 (CBCASUS10364416) were aligned using MultAlin (http://multalin.toulouse.inra.fr/multalin/) which showed the presence of single nucleotide polymorphisms (SNPs) A45G and C300T only in CBCASexpressed (FIG. 6). Additionally, it appears as no deposited CBCAS sequence on NCBI including whole genome assembly sequences contained either SNPs A45G or C300T showing CBCASexpressed is a novel CBCAS allele.

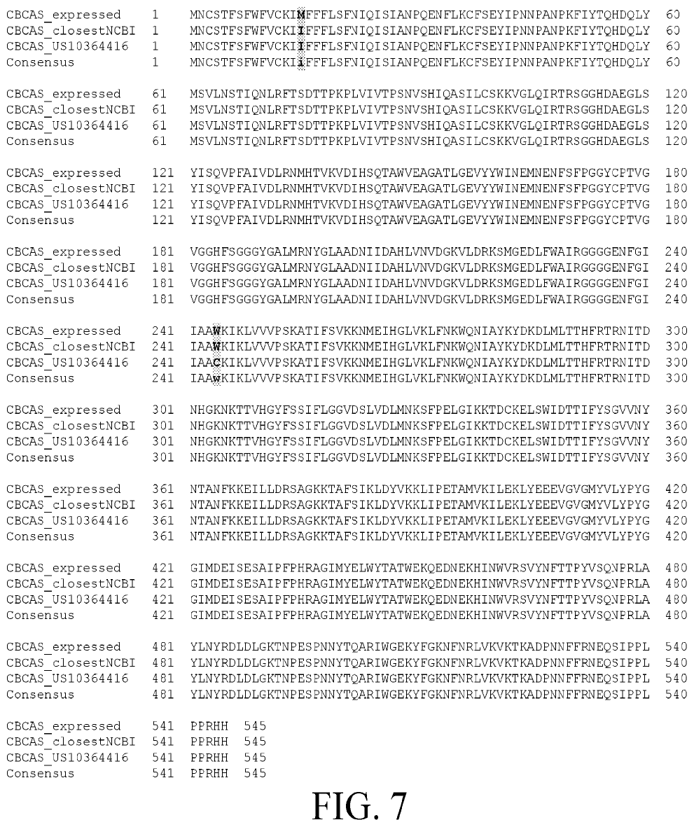

Amino acid sequences encoded by CBCASexpressed, CBCASclosestNCBI, and CBCASUS10364416 were aligned using MultAlin which showed the presence of amino acid change I15M only in CBCASexpressed (FIG. 7) which is located in the protein's signal sequence and not expected to alter enzymatic function.

### Example 3—Demonstration of CBCASexpressed Specificity to the Genome of Strain CGC1

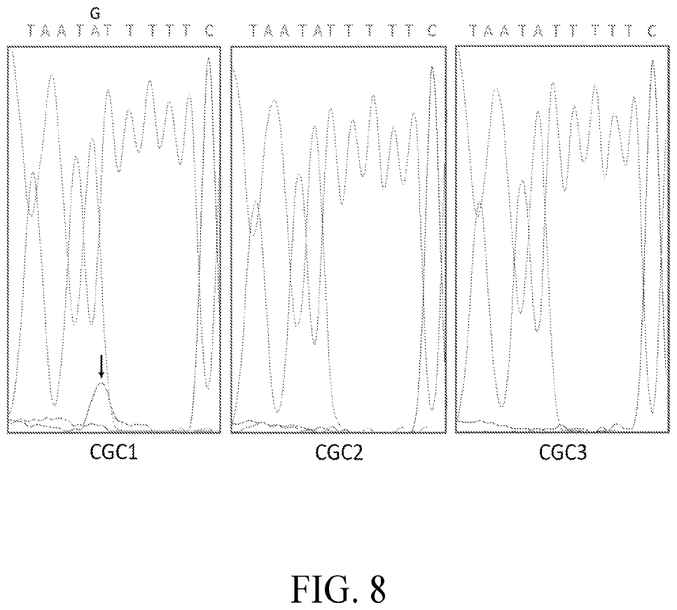

In order to determine if CBCASexpressed allele was specific to CGC1 and not present in other genotypes, PCR products for CBCAS amplified from gDNA template for CGC1, CGC2 and CGC3 with primers CBCAS ORF Forward and CBCAS 3′UTR Reverse (FIG. 5; right panel) were excised and DNA was purified. Sanger sequencing was performed with forward and reverse primers as well as the Internal Reverse sequencing primer. Results showed that dominant chromatogram peaks representing the CBCASclosestNCBI sequence were most prominent for all three strains, with multiple minor chromatogram peaks at various nucleotide positions in all strains indicating low-weighted SNPs. This result is indicative of the presence of multiple CBCAS alleles with some sequence divergence from CBCASclosestNCBI present in the genomes of all strains. An example is presented in FIG. 8 where a low weighted peak representing the SNP A45G of CBCASexpressed was detected only in CGC1 but not in CGC2 or CGC3. This data suggested that CBCASexpressed was indeed only present in the genome of CGC1 but not CGC2 or CGC3.

To investigate the possibility of multiple CBCAS alleles present in the genomes of the three tested strains, clonal allele analysis was performed. CBCAS coding sequences were amplified by PCR as described above using gDNA templates with primers CBCAS ORF Forward Gibson 5′-AGCAAGTTCTTCACTGTTGATACATAAATGAATTGCTCAACATTC-3′ (SEQ ID NO: 12) and CBCAS ORF Reverse Gibson 5′-GAGTTGTTGATTCAGAATTGTCGACGTAGATAATTAATGATGACGCG-3′ (SEQ ID NO: 13), amplification products were purified and cloned into the first multiple cloning site of plasmid vector pRI 201-AN (Takara Bio) using NEBuilder HiFi DNA Assembly Master Mix (New England Biolabs). Cloned plasmids were transformed into NEB 10-beta Competent E. coli and colonies were selected and grown overnight and plasmid DNA isolated. Full-length CBCAS sequences from cloned plasmids were Sanger sequenced with primers 35s Forward 5′-CTATCCTTCGCAAGACCCTTC-3′ (SEQ ID NO: 14) and MCS1 Reverse 5′-CAAACTTAAGCACACAAGCTAGC-3′ (SEQ ID NO: 15) and manually assembled. 22 clones for CGC1, 20 clones for CGC2, and 20 clones for CGC3 were sequenced.

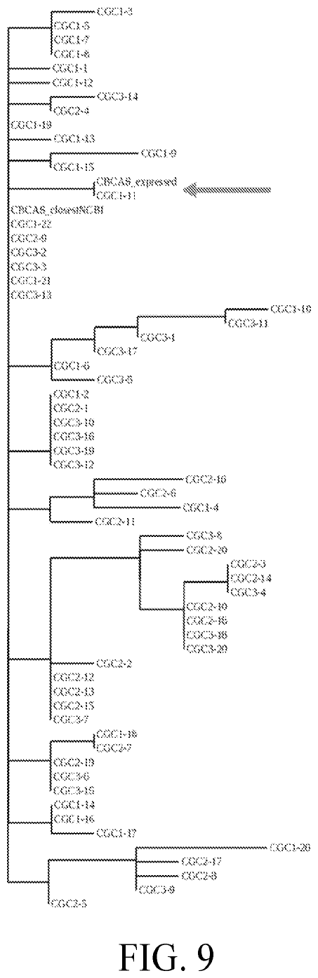

Results of CBCAS clonal sequencing showed 17/22 for CGC1, 16/20 for CGC2, and 13/20 CGC3 sequences were unique CBCAS alleles. A phylogeny of all clonal CBCAS sequences for all three strains, also with CBCASexpressed, rooted on the CBCASclosestNCBI sequence was made using the “one click” mode on phylogeny.fr (FIG. 9). In total across the three strains 36 unique CBCAS alleles with at least one SNP or single nucleotide insertion or deletion difference from CBCASclosestNCBI were found, and CBCASclosestNCBI was found in the genome all three strains. All alleles shared >99.5% sequence identity to each other and many alleles were found across genotypes. Importantly, an exact match for CBCASexpressed was only found in CGC1 (CGC1-11, FIG. 9), providing more evidence that CBCASexpressed is a rare allele only present in the CGC1 genome but not in CGC2 or CGC3.

Collectively these results show the presence of at least 13 to 17 unique CBCAS alleles are present in each genome of CGC1, CGC2, and CGC3. This is in accordance with the presence of tandem CBCAS duplication “cassettes” found in most strains through whole-genome sequencing (McKernan et al., 2020). CBCAS sequences were found to be flanked by putatively active retrotransposon elements which may lead to high genomic sequence duplication (Grassa et al., 2018). However, it is noted here that many more unique CBCAS alleles were found in this analysis than have been presented to date, and this is likely due to multiple sequence “collapse” to a single sequence during the genome assembly process from two or more highly similar, but unique, sequences. Nevertheless, it is striking that with the presence of at least 37 unique CBCAS alleles across the genomes of the three strains analyzed, that only CBCASexpressed was determined to be transcriptionally expressed in cannabis flower glandular trichomes (FIG. 5). This data provides even more evidence that CBCAS expression is rare in modern cannabis strains.

The reason that CBCASexpressed is transcriptionally expressed in glandular trichomes in contrast to the other 36 alleles found in this study was not investigated. Because the SNPs A45G and C300T unique to CBCASexpressed are found in the coding region they are not expected to alter expression. However, it is noted here that these SNPs may alter epigenetic markers and chromatin accessibility allowing for expression of CBCASexpressed relative to the other alleles found in this study. Another possibility is that CBCASexpressed is linked to a change in its promoter region which allows for gene expression of CBCASexpressed relative to the other alleles.

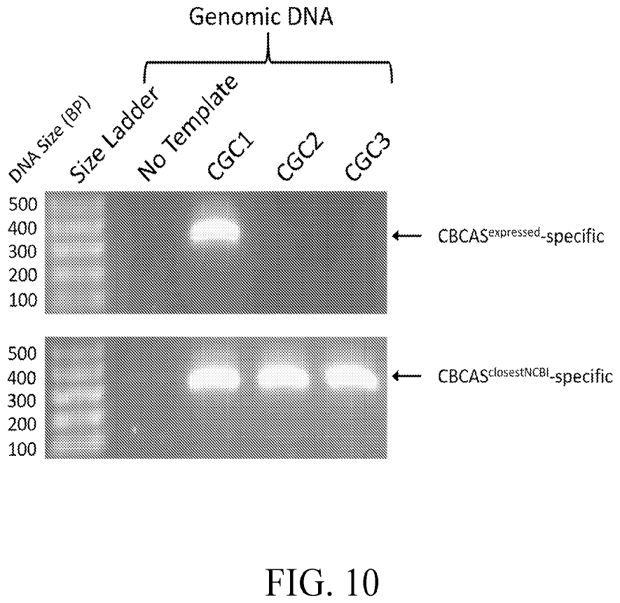

To provide more evidence that CBCASexpressed is only present in the genome of CGC1 but not CGC2 or CGC3, allele-specific PCR was used. For primers specific to CBCASexpressed, forward and reverse PCR primers terminating on SNPs A45G and C300T, respectively, in CBCASexpressed were developed. These primers were CBCASexpressed Forward 5′-CTCCTTTTGGTTTGTTTGCAAAATACTG-3′ (SEQ ID NO: 16) and CBCASexpressed Reverse 5′-CGAATCTGCAAACCAACTTTCGTA-3′ (SEQ ID NO: 17). For primers specific to CBCASclosestNCBI, forward and reverse PCR primers terminating on A45 and C300, respectively, in CBCASclosestNCBIwere developed. These primers were CBCASclosestNCBI Forward 5′-CTCCTTTTGGTTTGTTTGCAAAATAATA-3′ (SEQ ID NO: 18) and CBCASclosestNCBI Reverse 5′-CGAATCTGCAAACCAACTTTCTTG-3′ (SEQ ID NO: 19). PCR with these two primer sets was performed on gDNA templates from the three strains using GoTaq® enzyme (Promega®) with the following thermal cycler program: 1) 95° C. for 2 min; 2) 95° C. for 30 sec; 3) 49° C. for 30 sec; 4) 72° C. for 2 min (repeat 2-4 34×); 5) 72° C. for 5 min; and 6) Hold 4° C. PCR results are presented in FIG. 10. Results showed that although CBCASclosestNCBI was present in the genome of all three strains, CBCASexpressed was only detected in the genome of CGC1.

Collectively, the data presented here show that of at least 37 unique CBCAS alleles, only CBCASexpressed is expressed in cannabis flower glandular trichomes and that it is only present in the genome of CGC1 but not CGC2 or CGC3. These results further show that CBCASexpressed and the correlated ability to accumulate over 1% total CBC by dry weight is exceedingly rare in modern cannabis strains.

### Example 4—Demonstration of the Absence of PJC in Strain CGC1

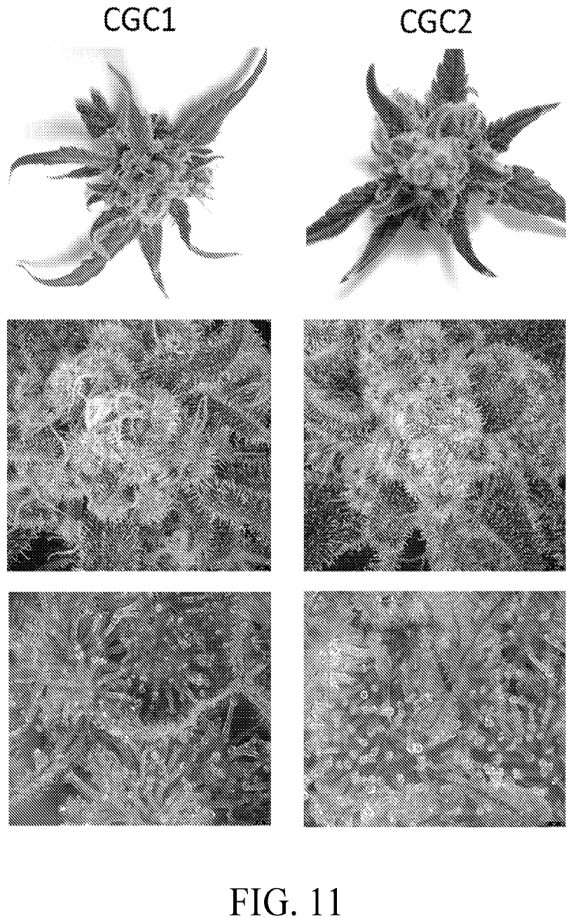

De Meijer et. al had previously bred a CBC-dominant cannabis strain that was genetically linked to the absence of stalked glandular trichomes during the course of flower development known as “prolonged juvenile characteristic” or PJC (E. P. M. De Meijer et al., 2009) (US Patent application 20110098348 and 20160360721). In order to determine if CBC accumulation in CGC1 was linked to a PJC, microscopic photographs were taken of CGC1 and CGC2 flowers and trichomes at 7 weeks post flower initiation using a Leica M205 FCA stereomicroscope equipped with a Leica DMC4500 digital camera. Results showed the near-complete absence of sessile (non-stalked) trichomes and that CGC1 and CGC2 had similar stalked glandular trichome density (FIG. 11). The abundance of stalked glandular trichomes at flower maturity firmly shows that CBCAS accumulation in CGC1 is not linked to PJC.

### Example 5—Demonstration that CGC1 is Heterozygous for CBCASexpressed

To determine if CBC-enriched strain CGC1 was heterozygous or homozygous for CBCASexpressed, it was crossed with a CBG-dominant strain (as described in PCT/US2021/041818, the entirety of which is incorporated herein by reference), which does not contain CBCASexpressed. Male flowers were induced on the genetically female CBG-dominant plant using foliar silver thiosulphate sprays at the beginning of the flowering period (Lubell & Brand, 2018). Pollen from the CBG-dominant plant was then used to fertilize a flowering CGC1 plant and F1 seeds from the cross were harvested at 10 weeks post-flower induction.

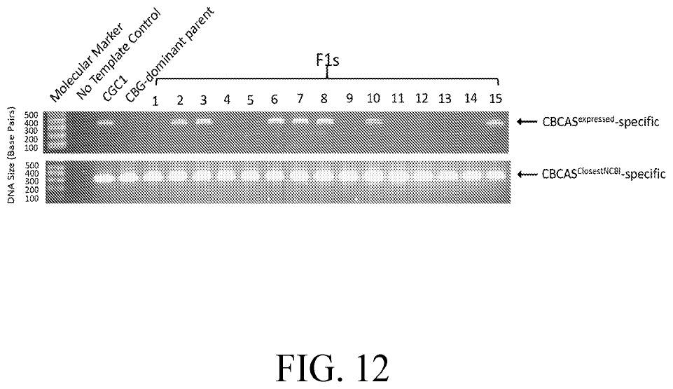

A number of 15 F1 seeds from the cross were germinated and established in rockwool (Grodan®) and young leaves were excised and used for gDNA extraction as described above. To investigate the genomic CBCASexpressed composition of the F1s, PCR was performed on F1 gDNA samples as well as gDNA isolated from the F1 parents using primers SEQ ID NO:16 and SEQ ID NO:17 for CBCASexpressed and SEQ ID NO: 18 and SEQ ID NO: 19 for CBCASclosestNCBI as described above (FIG. 12). CBCASclosestNCBI served as a PCR positive control since it was found in both parents. Results of the F1 progeny showed that the CBCASexpressed allele segregation followed the expected Mendelian inheritance ratio of 1:1 CBCASexpressed-positive:CBCASexpressed-negative progeny if CGC1 was heterozygous for CBCASexpressed. Specifically, from the 15 F1 progeny, 7 were CBCASexpressed-positive and 8 were CBCASexpressed-negative. A Chi-squared test of independence gave a value of 0.8 against an expected 7.5:7.5 distribution using the CHISQ.TEST function in Microsoft Excel (Table 2). This showed that CGC1 was heterozygous for CBCASexpressed.

TABLE 2
Expected and actual distribution and Chi-squared test value
of CBCASexpressed-positive and CBCASexpressed-negative
Fl progeny of CGC1 crossed with a CBG-dominant plant.

| | Expected | Actual | Chi Squared Test |
|---|---|---|---|
| CBCASexpressed-positive | 7.5 | 7 | 0.80 |
| CBCASexpressed-negative | 7.5 | 8 | |

### Example 6—Demonstration that Expression of CBCAS Allele Causes CBC Enrichment

To determine if genomic presence of CBCASexpressed was associated with CBC enrichment, CGC1 was self-pollenated by male induction using silver thiosulphate sprays and selfed (Sis) seeds were harvested as described above.

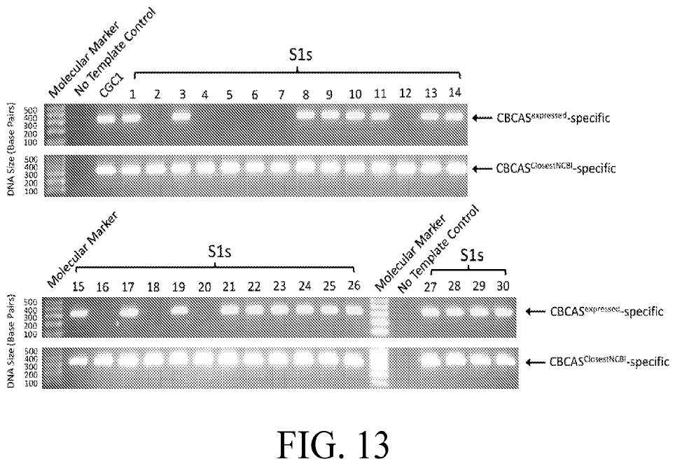

A number of 30 S1 seeds from the cross were germinated and established in rockwool (Grodan®) and young leaves were excised and used for gDNA extraction as described above. To investigate the genomic CBCASexpressed composition of the S1s, PCR was performed on S1 gDNA samples as well as gDNA isolated from the CGC1 parent using primers SEQ ID NO:16 and SEQ ID NO:17 for CBCASexpressed and SEQ ID NO: 18 and SEQ ID NO: 19 for CBCASclosestNCBI as described above (FIG. 13). CBCASclosestNCBI served as a PCR positive control since it was found in all gDNA samples. Results of the S1 progeny showed that the CBCASexpressed allele segregation again followed the expected Mendelian inheritance ratio of 3:1 CBCASexpressed-positive:CBCASexpressed-negative progeny if CGC1 was heterozygous for CBCASexpressed. Specifically, from the 30 S1 progeny, 21 were CBCASexpressed-positive and 9 were CBCASexpressed-negative. A Chi-squared test of independence gave a value of 0.55 against an expected 22.5.5:7.5 distribution using the CHISQ.TEST function in Microsoft Excel (Table 3). This again showed that CGC1 was heterozygous for CBCASexpressed.

TABLE 3
Expected and actual distribution and Chi-squared test
value of CBCASexpressed-positive and CBCASexpressed-
negative F1 progeny of self-pollenated CGC1.

| | Expected | Actual | Chi Squared Test |
|---|---|---|---|
| CBCASexpressed-positive | 22.5 | 21 | 0.55 |
| CBCASexpressed-negative | 7.5 | 9 | |

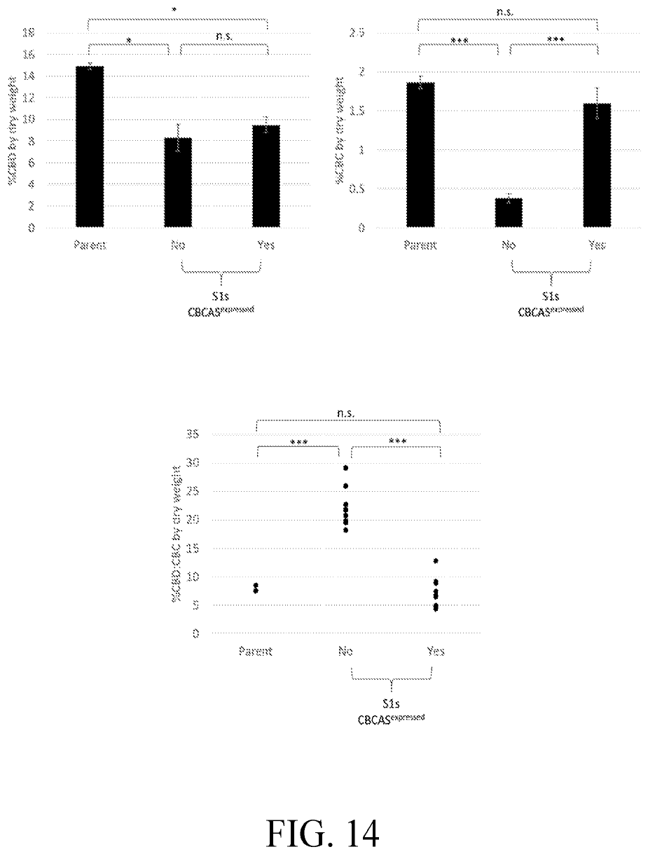

The presence of both CBCASexpressed-positive and CBCASexpressed-negative populations of CGC1 S1 progeny allowed the unbiased genetic determination if genomic presence of CBCASexpressed was associated with CBC enrichment. Two replicates of parent CGC1 and S1 progeny number 1-26 from FIG. 13 were clonally propagated, established, and flowered as described above. During propagation or early flowering, 4 CBCASexpressed-negative progenies died and were unable to be included in chemotype analysis. This left the 2 CGC1 parental replicates, 13 CBCASexpressed-positive S1 progenies and 9 CBCASexpressed-negative progenies. Flowers were harvested 7 weeks post flower induction, prepared, and cannabinoid content was analyzed via HPLC as described above. Results for total cannabinoid content by dry weight in flowers for CBD, CBC, and CBD:CBC ratios are depicted in FIG. 14 and S1 progeny were categorized into CBCASexpressed-positive and CBCASexpressed-negative based on allele screening results of FIG. 13. In FIG. 14, the left and middle panel represent population means and standard error and each dot in the right panel represents a data point from an individual plant.

Results in FIG. 14 showed that on average CGC1 had significantly more total CBD than either CBCASexpressed-positive and CBCASexpressed-negative progeny populations, possibly due to inbreeding depression from selfing (Kurtz et al., 2020). However, there was no significant difference between the mean CBD content between CBCASexpressed-positive and CBCASexpressed-negative progeny populations showing that presence or absence of genomic CBCASexpressed was not correlated with CBDAS accumulation.

In stark contrast, results showed that on average CGC1 had significantly more total CBC than CBCASexpressed-negative progeny. In addition, CBCASexpressed-positive progeny had significantly higher average total CBC than CBCASexpressed-negative progeny. Further, average total CBC was not significantly different between CGC1 and CBCASexpressed-positive progeny despite CGC1 having significantly more total CBD. This result suggests that CBCASexpressed-positve progeny homozygous for the allele may accumulate higher proportional amounts of CBCAS than heterozygous parent CGC1, although the assay described in FIG. 13 is not capable of determining zygosity. Indeed, 4 CBCASexpressed-positve progenies had higher total CBC than both CGC1 replicates, up to 2.54%, despite having less total CBD than both CGC1 replicates. No CBCASexpressed-negative progeny accumulated higher than 0.65% CBC, whereas only one CBCASexpressed-positive plant in this trial including parents tested under 0.65% CBC at 0.63% total CBC, likely because it had low CBD content as well at 5.8%. Together, these data clearly show that CBC-enrichment is associated with the genomic presence of CBCASexpressed.

Because total CBD ranged highly between all progeny, between 3.33 and 12.87%, the total CBD:CBC ratio is a better comparison for CBC enrichment which controls for total cannabinoid content. Per plant total CBD:CBC ratios are presented in FIG. 14 right panel as individual points. Results showed that all CBCASexpressed-positive samples, including parent CGC1 and CBCASexpressed-positive progeny had lower CBD:CBC ratios than any CBCASexpressed-negative progeny. This clearly shows that genomic CBCASexpressed is linked to CBC-enrichment and provides firm evidence that the transcriptionally expressed CBCAS allele described here causes CBC enrichment.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

### BIBLIOGRAPHY

Appendino, G., Gibbons, S., Giana, A., Pagani, A., Grassi, G., Stavri, M., Smith, E., & Rahman, M. M. (2008). Antibacterial cannabinoids from Cannabis sativa: A structure-activity study. Journal of Natural Products. https://doi.org/10.1021/np8002673
Baker, P. B., Gough, T. A., & Taylor, B. J. (1983). The physical and chemical features of Cannabis plants grown in the United Kingdom of Great Britain and Northern Ireland from seeds of known origin. Part II: second generation studies. Bulletin on Narcotics.
Braich, S., Baillie, R. C., Jewell, L. S., Spangenberg, G. C., & Cogan, N. O. I. (2019). Generation of a Comprehensive Transcriptome Atlas and Transcriptome Dynamics in Medicinal Cannabis. Scientific Reports. https://doi.org/10.1038/s41598-019-53023-6
Davis, W. M., & Hatoum, N. S. (1983). Neurobehavioral actions of cannabichromene and interactions with Δ9-tetrahydrocannabinol. General Pharmacology. https://doi.org/10.1016/0306-3623(83)90004-6
De Meijer, E. P. M., Hammond, K. M., & Micheler, M. (2009). The inheritance of chemical phenotype in Cannabis sativa L. (III): Variation in cannabichromene proportion. Euphytica. https://doi.org/10.1007/s10681-008-9787-1
De Meijer, Etienne P. M., Bagatta, M., Carboni, A., Crucitti, P., Moliterni, V. M. C., Ranalli, P., & Mandolino, G. (2003). The inheritance of chemical phenotype in Cannabis sativa L. Genetics.
DeLong, G. T., Wolf, C. E., Poklis, A., & Lichtman, A. H. (2010). Pharmacological evaluation of the natural constituent of Cannabis sativa, cannabichromene and its modulation by Δ9-tetrahydrocannabinol. Drug and Alcohol Dependence. https://doi.org/10.1016/i.drugalcdep.2010.05.019
Di Marzo, V., & Piscitelli, F. (2015). The Endocannabinoid System and its Modulation by Phytocannabinoids. In Neurotherapeutics. https://doi.org/10.1007/s13311-015-0374-6
El-Alfy, A. T., Ivey, K., Robinson, K., Ahmed, S., Radwan, M., Slade, D., Khan, I., ElSohly, M., & Ross, S. (2010). Antidepressant-like effect of Δ9-tetrahydrocannabinol and other cannabinoids isolated from Cannabis sativa L. Pharmacology Biochemistry and Behavior. https://doi.org/10.1016/j.pbb.2010.03.004
ElSohly, M. A., & Slade, D. (2005). Chemical constituents of marijuana: The complex mixture of natural cannabinoids. Life Sciences. https://doi.org/10.1016/j.lfs.2005.09.011
Gagne, S. J., Stout, J. M., Liu, E., Boubakir, Z., Clark, S. M., & Page, J. E. (2012). Identification of olivetolic acid cyclase from Cannabis sativa reveals a unique catalytic route to plant polyketides. Proceedings of the National Academy of Sciences of the United States of America. https://doi.org/10.1073/pnas.1200330109
Galletta, M., Reekie, T. A., Nagalingam, G., Bottomley, A. L., Harry, E. J., Kassiou, M., & Triccas, J. A. (2020). Rapid antibacterial activity of cannabichromenic acid against methicillin-resistant Staphylococcus aureus. Antibiotics. https://doi.org/10.3390/antibiotics9080523
Grassa, C. J., Wenger, J. P., Dabney, C., Poplawski, S. G., Motley, S. T., Michael, T. P., Schwartz, C. J., & Weiblen, G. D. (2018). A complete Cannabis chromosome assembly and adaptive admixture for elevated cannabidiol (CBD) content. BioRxiv. https://doi.org/10.1101/458083
Hanus̆, L. O., Meyer, S. M., Munoz, E., Taglialatela-Scafati, O., & Appendino, G. (2016). Phytocannabinoids: A unified critical inventory. In Natural Product Reports. https://doi.org/10.1039/c6np00074f
Hillig, K. W., & Mahlberg, P. G. (2004). A chemotaxonomic analysis of cannabinoid variation in Cannabis (Cannabaceae). American Journal of Botany. https://doi.org/10.3732/ajb.91.6.966
Izzo, A. A., Capasso, R., Aviello, G., Borrelli, F., Romano, B., Piscitelli, F., Gallo, L., Capasso, F., Orlando, P., & Di Marzo, V. (2012). Inhibitory effect of cannabichromene, a major non-psychotropic cannabinoid extracted from Cannabis sativa, on inflammation-induced hypermotility in mice. British Journal of Pharmacology. https://doi.org/10.1111/j.1476-5381.2012.01879.x
Laverty, K. U., Stout, J. M., Sullivan, M. J., Shah, H., Gill, N., Holbrook, L., Deikus, G., Sebra, R., Hughes, T. R., Page, J. E., & Van Bakel, H. (2019). A physical and genetic map of Cannabis sativa identifies extensive rearrangements at the THC/CBD acid synthase loci. Genome Research. https://doi.org/10.1101/gr.242594.118
Lewis, M. A., Russo, E. B., & Smith, K. M. (2018). Pharmacological Foundations of Cannabis Chemovars. Planta Medica. https://doi.org/10.1055/s-0043-122240
Livingston, S. J., Quilichini, T. D., Booth, J. K., Wong, D. C. J., Rensing, K. H., Laflamme-Yonkman, J., Castellarin, S. D., Bohlmann, J., Page, J. E., & Samuels, A. L. (2020). Cannabis glandular trichomes alter morphology and metabolite content during flower maturation. Plant Journal. https://doi.org/10.1111/tpj.14516
Mahlberg, P. G., & Eun, S. K. (2004). Accumulation of cannabinoids in glandular trichomes of Cannabis (Cannabaceae). Journal of Industrial Hemp. https://doi.org/10.1300/J237v09n01_04
Maione, S., Piscitelli, F., Gatta, L., Vita, D., De Petrocellis, L., Palazzo, E., De Novellis, V., & Di Marzo, V. (2011). Non-psychoactive cannabinoids modulate the descending pathway of antinociception in anaesthetized rats through several mechanisms of action. British Journal of Pharmacology. https://doi.org/10.1111/j.1476-5381.2010.01063.x
McGarvey, P., Huang, J., McCoy, M., Orvis, J., Katsir, Y., Lotringer, N., Nesher, I., Kavarana, M., Sun, M., Peet, R., Meiri, D., & Madhavan, S. (2020). De novo assembly and annotation of transcriptomes from two cultivars of Cannabis sativa with different cannabinoid profiles. Gene. https://doi.org/10.1016/j.gene.2020.145026
McKernan, K. J., Helbert, Y., Kane, L. T., Ebling, H., Zhang, L., Liu, B., Eaton, Z., McLaughlin, S., Kingan, S., Baybayan, P., Concepcion, G., Jordan, M., Riva, A., Barbazuk, W., & Harkins, T. (2020). Sequence and annotation of 42 cannabis genomes reveals extensive copy number variation in cannabinoid synthesis and pathogen resistance genes. BioRxiv. https://doi.org/10.1101/2020.01.03.894428
McPartland, J. M., & Guy, G. W. (2017). Models of Cannabis Taxonomy, Cultural Bias, and Conflicts between Scientific and Vernacular Names. Botanical Review. https://doi.org/10.1007/s12229-017-9187-0
McPartland, J. M., & Small, E. (2020). A classification of endangered high-THC Cannabis (Cannabis sativa subsp. indica) domesticates and their wild relatives. PhytoKeys. https://doi.org/10.3897/PHYTOKEYS.144.46700
Morimoto, S., Komatsu, K., Taura, F., & Shoyama, Y. (1997). Enzymological evidence for cannabichromenic acid biosynthesis. Journal of Natural Products. https://doi.org/10.1021/np970210v
Morimoto, Satoshi, Komatsu, K., Taura, F., & Shoyama, Y. (1998). Purification and characterization of cannabichromenic acid synthase from Cannabis sativa. Phytochemistry. https://doi.org/10.1016/S0031-9422(98)00278-7
Pollastro, F., Caprioglio, D., Prete, D. Del, Rogati, F., Minassi, A., Taglialatela-Scafati, O., Munoz, E., & Appendino, G. (2018). Cannabichromene. In Natural Product Communications. https://doi.org/10.1177/1934578x1801300922
Romano, B., Borrelli, F., Fasolino, I., Capasso, R., Piscitelli, F., Cascio, M. G., Pertwee, R. G., Coppola, D., Vassallo, L., Orlando, P., Di Marzo, V., & Izzo, A. A. (2013). The cannabinoid TRPA1 agonist cannabichromene inhibits nitric oxide production in macrophages and ameliorates murine colitis. British Journal of Pharmacology. https://doi.org/10.1111/bph.12120
ROWAN, M. G., & FAIRBAIRN, J. W. (1977). Cannabinoid patterns in seedlings of Cannabis sativa L. and their use in the determination of chemical race. Journal of Pharmacy and Pharmacology. https://doi.org/10.1111/j.2042-7158.1977.tb11375.x
Schultes, R. E., Schultes, R. E., Klein, W. M., Plowman, T., & Lockwood, T. E. (1974). Cannabis: an example of taxonomic neglect. Botanical Museum Leaflets, Harvard University.
Shinjyo, N., & Di Marzo, V. (2013). The effect of cannabichromene on adult neural stem/progenitor cells. Neurochemistry International. https://doi.org/10.1016/j.neuint.2013.08.002
Shoyama, Y., Yagi, M., Nishioka, I., & Yamauchi, T. (1975). Biosynthesis of cannabinoid acids. Phytochemistry. https://doi.org/10.1016/S0031-9422(00)91096-3
Small, E. (1972). Interfertility and chromosomal uniformity in Cannabis. Canadian Journal of Botany. https://doi.org/10.1139/b72-248
Small, E., & Beckstead, H. D. (1973). Cannabinoid phenotypes in Cannabis sativa. Nature. https://doi.org/10.1038/245147a0
Small, E., & Cronquist, A. (1976). A PRACTICAL AND NATURAL TAXONOMY FOR CANNABIS. TAXON. https://doi.org/10.2307/1220524
Stone, N. L., Murphy, A. J., England, T. J., & O'Sullivan, S. E. (2020). A systematic review of minor phytocannabinoids with promising neuroprotective potential. In British Journal of Pharmacology. https://doi.org/10.1111/bph.15185
Turner, C. E., & Elsohly, M. A. (1981). Biological activity of cannabichromene, its homologs and isomers. Journal of Clinical Pharmacology. https://doi.org/10.1002/j.1552-4604.1981.tb02606.x
Turner, C. E., Hadley, K. W., Holley, J. H., Billets, S., & Mole, M. L. (1975). Constituents of cannabis sativa, L. VIII: Possible biological application of a new method to separate cannabidiol and cannabichromene. Journal of Pharmaceutical Sciences. https://doi.org/10.1002/jps.2600640517
Turner, Carlton E., & Hadley, K. (1973a). Constituents of Cannabis sativa L. II: Absence of cannabidiol in an african variant. Journal of Pharmaceutical Sciences. https://doi.org/10.1002/jps.2600620214
Turner, Carlton E., & Hadley, K. W. (1973b). Constituents of Cannabis sativa L. III: Clear and discrete separation of cannabidiol and cannabichromene. Journal of Pharmaceutical Sciences. https://doi.org/10.1002/jps.2600620706
Vergara, D., Gaudino, R., Blank, T., & Keegan, B. (2020). Modeling cannabinoids from a large-scale sample of Cannabis sativa chemotypes. PLoS ONE. https://doi.org/10.1371/journal.pone.0236878
Vogelmann, A. F., Turner, J. C., & Mahlberg, P. G. (1988). Cannabinoid composition in seedlings compared to adult plants of cannabis sativa. Journal of Natural Products. https://doi.org/10.1021/np50060a004
Wirth, P. W., Sue Watson, E., ElSohly, M., Turner, C. E., & Murphy, J. C. (1980). Anti-inflammatory properties of cannabichromene. Life Sciences. https://doi.org/10.1016/0024-3205(80)90631-1
Kurtz, L. E., Mahoney, J. D., Brand, M. H., & Lubell-Brand, J. D. (2020). Comparing genotypic and phenotypic variation of selfed and outcrossed progeny of hemp. HortScience. https://doi.org/10.21273/HORTSCI15061-20
Lubell, J. D., & Brand, M. H. (2018). Foliar sprays of silver thiosulfate produce male flowers on female hemp plants. HortTechnology. https://doi.org/10.21273/HORTTECH04188-18

1. A Cannabis plant, plant part, tissue or cell thereof, wherein the Cannabis plant, plant part, tissue or cell thereof has a cannabichromenic acid (CBCA) and/or cannabichromene (CBC) content greater than about 1% of total dry flower weight.

2.-6. (canceled)

7. The Cannabis plant, plant part, tissue or cell thereof of claim 1, further having a total cannabinoid content of between about 3% and about 30% of total dry flower weight.

8.-10. (canceled)

11. The Cannabis plant, plant part, tissue or cell thereof of claim 1, further having a cannabidiol (CBD):CBC ratio is between about 15:1 and about 1:2000.

12. (canceled)

13. The Cannabis plant, plant part, tissue or cell thereof of claim 1, wherein the Cannabis plant is in a flowering state.

14. The Cannabis plant, plant part, tissue or cell thereof of claim 1, wherein the cannabis plant exhibits stalked glandular trichomes.

15. The Cannabis plant, plant part, tissue or cell thereof of claim 1, wherein the cannabis plant does not exhibit prolonged juvenile characteristic (PJC).

16. The Cannabis plant, plant part, tissue or cell thereof of claim 1, wherein the Cannabis plant, plant part, tissue or cell thereof is a hybrid Cannabis plant, plant part, tissue or cell thereof.

17. The Cannabis plant, plant part, tissue or cell thereof of claim 16, wherein the hybrid Cannabis plant, plant part, tissue or cell thereof is an asexual clone.

18.-59. (canceled)

60. A Cannabis plant comprising a variant cannabichromenic acid synthase (CBCAS) allele, wherein the expression-altering variant CBCAS allele encodes for CBCAS.

61. The Cannabis plant of claim 60, comprising a nucleic acid sequence as set forth in SEQ ID NO: 8.

62. The Cannabis plant of claim 60, further having a cannabichromenic acid (CBCA) and/or cannabichromene (CBC) content greater than about 1% of total dry flower weight.

63. The Cannabis plant of claim 60, further having a total cannabinoid content of between about 3% and about 30% of total dry flower weight.

64. The Cannabis plant of claim 60, wherein the cannabis plant does not exhibit prolonged juvenile characteristic (PJC).

65. A Cannabis plant comprising an expression-altering variant cannabichromenic acid synthase (CBCAS) allele, wherein the plant expresses a CBCAS transcript.

66. The Cannabis plant of claim 65, comprising a nucleic acid sequence as set forth in SEQ ID NO: 8.

67. The Cannabis plant of claim 65, further having a cannabichromenic acid (CBCA) and/or cannabichromene (CBC) content greater than about 1% of total dry flower weight.

68. The Cannabis plant of claim 65, further having a total cannabinoid content of between about 3% and about 30% of total dry flower weight.

69. The Cannabis plant of claim 65, wherein the cannabis plant does not exhibit prolonged juvenile characteristic (PJC).

70. The Cannabis plant, plant part, tissue or cell thereof of claim 1, further having a cannabidiol (CBD):CBC ratio is between about 15:1 and about 1:1.

71. The Cannabis plant, plant part, tissue or cell thereof of claim 1, further having a cannabidiol (CBD):CBC ratio is between about 1:1 and about 1:25.
